SEQUENCE LISTING (111 sequences)

SEQ ID NO: 1 - protein, 37 aa, Unknown
  Parabuthus transvaalicus toxin
QMDMRCSASVECKQKCLKAIGSIFGKCMNKKCKCYPR

SEQ ID NO: 2 - protein, 37 aa, Artificial Sequence
  Modified SEQ ID NO 1
NMDMRCKASVECKQKCLKAIGSIFGKCMNKKCKCYPR

SEQ ID NO: 3 - protein, 37 aa, Artificial Sequence
  Modified SEQ ID NO 1
NMDMRCSASRECKQKCLKAIGSIFGKCMNKKCKCYPR

SEQ ID NO: 4 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
NXDXRCRASVECKQKCLKAIGSIFGKCXNKKCKCYPR

SEQ ID NO: 5 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
NXDXRCSHSVECKQKCLKAIGSIFGKCXNKKCKCYPR

SEQ ID NO: 6 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
NXDXRCSASKECKQKCLKAIGSIFGKCXNKKCKCYPR

SEQ ID NO: 7 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXEXRCFASVECKQKCLAAIGSIFGKCXNKKCKCYPR

SEQ ID NO: 8 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXEXRCSYSVECKQKCLAAIGSIFGKCXNKKCKCYPR

SEQ ID NO: 9 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXEXRCSAFVECKQKCLAAIGSIFGKCXNKKCKCYPR

SEQ ID NO: 10 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (7)..(7): Xaa is 2,4-diaminobutanoyl
  MISC_FEATURE (28)..(28): Xaa is norleucine
PXEXRCXASVECKQKCLAAIGSIFGKCXNKKCKCYPR

SEQ ID NO: 11 - protein, 37 aa, Artificial Sequence; identical to SEQ ID NO: 10
  Modified SEQ ID NO 1
  MISC_FEATURE (2)..(2): Xaa is norleucine
  MISC_FEATURE (4)..(4): Xaa is norleucine
  MISC_FEATURE (7)..(7): Xaa is ornithine
  MISC_FEATURE (28)..(28): Xaa is norleucine

SEQ ID NO: 12 - protein, 37 aa, Artificial Sequence; identical to SEQ ID NO: 10
  Modified SEQ ID NO 1
  MISC_FEATURE (2)..(2): Xaa is norleucine
  MISC_FEATURE (4)..(4): Xaa is norleucine
  MISC_FEATURE (7)..(7): Xaa is 2-amino-3-guanidinopropionyl
  MISC_FEATURE (28)..(28): Xaa is norleucine

SEQ ID NO: 13 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXEXRCHASVECKQKCLAAIGSIFGKCXNKKCKCYPR

SEQ ID NO: 14 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXEXRCYASVECKQKCLAAIGSIFGKCXNKKCKCYPR

SEQ ID NO: 15 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (9)..(9): Xaa is ornithine
  MISC_FEATURE (28)..(28): Xaa is norleucine
PXEXRCSAXVECKQKCLAAIGSIFGKCXNKKCKCYPR

SEQ ID NO: 16 - protein, 37 aa, Artificial Sequence; identical to SEQ ID NO: 10
  Modified SEQ ID NO 1
  MISC_FEATURE (2)..(2): Xaa is norleucine
  MISC_FEATURE (4)..(4): Xaa is norleucine
  MISC_FEATURE (7)..(7): Xaa is 2,3-diaminopropanoyl
  MISC_FEATURE (28)..(28): Xaa is norleucine

SEQ ID NO: 17 - protein, 37 aa, Artificial Sequence; identical to SEQ ID NO: 15
  Modified SEQ ID NO 1
  MISC_FEATURE (2)..(2): Xaa is norleucine
  MISC_FEATURE (4)..(4): Xaa is norleucine
  MISC_FEATURE (9)..(9): Xaa is 2,3-diaminopropanoyl
  MISC_FEATURE (28)..(28): Xaa is norleucine

SEQ ID NO: 18 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (7)..(7): Xaa is 2,4-diaminobutanoyl
  MISC_FEATURE (28)..(28): Xaa is norleucine
PXEXRCXASVECAQSCLAAIGSIFGKCXNGKCKCYPR

SEQ ID NO: 19 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXEXRCRASVECAQSCLAAIGSIFGKCXNGKCKCYPR

SEQ ID NO: 20 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXEXRCSASRECAQSCLAAIGSIFGKCXNGKCKCYPR

SEQ ID NO: 21 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXEXRCSASVECKQKCLAAIGSIFGKCXNKKCKCYPR

SEQ ID NO: 22 - protein, 37 aa, Artificial Sequence
  Modified SEQ ID NO 1
QMDMRCSASVECKQKCLKAIGRGFGKCMNKKCKCYPR

SEQ ID NO: 23 - protein, 37 aa, Artificial Sequence
  Modified SEQ ID NO 1
NMDMRCSASVECKQKCLKAIGSIFGKCMNKKCKCYPR

SEQ ID NO: 24 - protein, 36 aa, Artificial Sequence
  Modified SEQ ID NO 1
MDMRCSASVECKQKCLKAIGSIFGKCMNKKCKCYPR

SEQ ID NO: 25 - protein, 37 aa, Artificial Sequence
  Modified SEQ ID NO 1
NIDMRCSASVECKQKCLKAIGSIFGKCMNKKCKCYPR

SEQ ID NO: 26 - protein, 37 aa, Artificial Sequence
  Modified SEQ ID NO 1
NMEMRCSASVECKQKCLKAIGSIFGKCMNKKCKCYPR

SEQ ID NO: 27 - protein, 37 aa, Artificial Sequence
  Modified SEQ ID NO 1
  MISC_FEATURE (28)..(28): Xaa is norleucine
NMDMRCSASVECKQKCLKAIGSIFGKCXNKKCKCYPR

SEQ ID NO: 28 - protein, 37 aa, Artificial Sequence
  Modified SEQ ID NO 1
NMDMRCSASVECKVKCLKAIGSIFGKCMNKKCKCYPR

SEQ ID NO: 29 - protein, 37 aa, Artificial Sequence
  Modified SEQ ID NO 1
NMDMRCSASVECKQLCLKAIGSIFGKCMNKKCKCYPR

SEQ ID NO: 30 - protein, 37 aa, Artificial Sequence
  Modified SEQ ID NO 1
NMDMRCSASVECKQKCKKAIGSIFGKCMNKKCKCYPR

SEQ ID NO: 31 - protein, 37 aa, Artificial Sequence
  Modified SEQ ID NO 1
NMDMRCSASVECKQKCLDAIGSIFGKCMNKKCKCYPR

SEQ ID NO: 32 - protein, 37 aa, Artificial Sequence
  Modified SEQ ID NO 1
NMDMRCSASVECKQKCLKAIRSIFGKCMNKKCKCYPR

SEQ ID NO: 33 - protein, 37 aa, Artificial Sequence
  Modified SEQ ID NO 1
NMDMRCSASVECKQKCLKAIESIFGKCMNKKCKCYPR

SEQ ID NO: 34 - protein, 41 aa, Artificial Sequence
  Modified SEQ ID NO 1
NMDMRCSASVECKQKCLKAIGSIFGKCMNKKCKCYPRRRTA

SEQ ID NO: 35 - protein, 37 aa, Artificial Sequence
  Modified SEQ ID NO 1
NMDMRCSISVECKQKCLKAIGSIFGKCMNKKCKCYPR

SEQ ID NO: 36 - protein, 37 aa, Artificial Sequence
  Modified SEQ ID NO 1
NMDMRCSASVQCKQKCLKAIGSIFGKCMNKKCKCYPR

SEQ ID NO: 37 - protein, 37 aa, Artificial Sequence
  Modified SEQ ID NO 1
NMDMRCSASVECLQKCLKAIGSIFGKCMNKKCKCYPR

SEQ ID NO: 38 - protein, 37 aa, Artificial Sequence
  Modified SEQ ID NO 1
NMDMRCSASVECAQKCLKAIGSIFGKCMNKKCKCYPR

SEQ ID NO: 39 - protein, 37 aa, Artificial Sequence
  Modified SEQ ID NO 1
NMDMRCSASVECKEKCLKAIGSIFGKCMNKKCKCYPR

SEQ ID NO: 40 - protein, 37 aa, Artificial Sequence
  Modified SEQ ID NO 1
NMDMRCSASVECKLKCLKAIGSIFGKCMNKKCKCYPR

SEQ ID NO: 41 - protein, 37 aa, Artificial Sequence
  Modified SEQ ID NO 1
NMDMRCSASVECKQKCLKAIHSIFGKCMNKKCKCYPR

SEQ ID NO: 42 - protein, 37 aa, Artificial Sequence
  Modified SEQ ID NO 1
NMDMRCSASVECKQKCLKAIGSKFGKCMNKKCKCYPR

SEQ ID NO: 43 - protein, 37 aa, Artificial Sequence
  Modified SEQ ID NO 1
NMDMRCSASVECKQKCLKAIGSRFGKCMNKKCKCYPR

SEQ ID NO: 44 - protein, 37 aa, Artificial Sequence
  Modified SEQ ID NO 1
NMDMRCSASVECKQKCLKAIGSIFGKCMNGKCKCYPR

SEQ ID NO: 45 - protein, 37 aa, Artificial Sequence
  Modified SEQ ID NO 1
NMDMRCSASVECKQKCLKAIGSIFGKCMNKKCHCYPR

SEQ ID NO: 46 - protein, 37 aa, Artificial Sequence
  Modified SEQ ID NO 1
NMDMRCSASVECKQKCLKAIGSIFGKCMNKKCVCYPR

SEQ ID NO: 47 - protein, 37 aa, Artificial Sequence
  Modified SEQ ID NO 1
  MISC_FEATURE (2)..(2): Xaa is norleucine
  MISC_FEATURE (4)..(4): Xaa is norleucine
NXDXRCSASVECKQKCLKAIGSIFGKCMNKKCKCYPR

SEQ ID NO: 48 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
NXDXRCSASVECKQKCLKAIGSIFGKCXNKKCKCYPR

SEQ ID NO: 49 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXDXRCSASVECKQKCLKAIGSIFGKCXNKKCKCYPR

SEQ ID NO: 50 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
NXDXRCSASVECEQKCLKAIGSIFGKCXNKKCKCYPR

SEQ ID NO: 51 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
NXDXRCSASVECQQKCLKAIGSIFGKCXNKKCKCYPR

SEQ ID NO: 52 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
NXDXRCSASVECKKKCLKAIGSIFGKCXNKKCKCYPR

SEQ ID NO: 53 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
NXSXRCSASVECKQKCLKAIGSIFGKCXNKKCKCYPR

SEQ ID NO: 54 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
NXDXRCSASVECKQSCLKAIGSIFGKCXNKKCKCYPR

SEQ ID NO: 55 - protein, 36 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (27)..(27): Xaa is norleucine
NXDXRCSASVECKQKCKAIGSIFGKCXNKKCKCYPR

SEQ ID NO: 56 - protein, 37 aa, Artificial Sequence
  Modified SEQ ID NO 1
NMDMRCSASVECKQKCYKAIGSIFGKCMNKKCKCYPR

SEQ ID NO: 57 - protein, 37 aa, Artificial Sequence
  Modified SEQ ID NO 1
NMDMRCSASVECKQKCRKAIGSIFGKCMNKKCKCYPR

SEQ ID NO: 58 - protein, 37 aa, Artificial Sequence
  Modified SEQ ID NO 1
NMDMRCSASVECKQKCLAAIGSIFGKCMNKKCKCYPR

SEQ ID NO: 59 - protein, 37 aa, Artificial Sequence
  Modified SEQ ID NO 1
NMDMRCSASVECKQKCLYAIGSIFGKCMNKKCKCYPR

SEQ ID NO: 60 - protein, 36 aa, Artificial Sequence
  Modified SEQ ID NO 1
NMDMRCSASVECKQKCLAIGSIFGKCMNKKCKCYPR

SEQ ID NO: 61 - protein, 37 aa, Artificial Sequence
  Modified SEQ ID NO 1
NMDMRCSASVECKQKCLKYIGSIFGKCMNKKCKCYPR

SEQ ID NO: 62 - protein, 37 aa, Artificial Sequence
  Modified SEQ ID NO 1
NMDMRCSASVECKQKCLKRIGSIFGKCMNKKCKCYPR

SEQ ID NO: 63 - protein, 37 aa, Artificial Sequence
  Modified SEQ ID NO 1
NMDMRCSASVECKQKCLKAYGSIFGKCMNKKCKCYPR

SEQ ID NO: 64 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
NXDXRCSASVECKQKCLKAIGSPFGKCXNKKCKCYPR

SEQ ID NO: 65 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
HXDXRCSASVECKQKCLKAIGSIFGKCXNKKCKCYPR

SEQ ID NO: 66 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
YXDXRCSASVECKQKCLKAIGSIFGKCXNKKCKCYPR

SEQ ID NO: 67 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
SXDXRCSASVECKQKCLKAIGSIFGKCXNKKCKCYPR

SEQ ID NO: 68 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
VXDXRCSASVECKQKCLKAIGSIFGKCXNKKCKCYPR

SEQ ID NO: 69 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (9)..(9): Xaa is alpha-aminobutyric acid
SXDXRCSAXVECKQKCLKAIGSIFGKCMNKKCKCYPR

SEQ ID NO: 70 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (26)..(26): Lys is homo-lysine
SXDXRCSASVECKQKCLKAIGSIFGKCMNKKCKCYPR

SEQ ID NO: 71 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (35)..(35): Xaa is 4-amino-phenylalanine
SXDXRCSASVECKQKCLKAIGSIFGKCMNKKCKCXPR

SEQ ID NO: 72 - protein, 37 aa, Artificial Sequence
  Modified SEQ ID NO 1
  MISC_FEATURE (2)..(2): Xaa is norleucine
  MISC_FEATURE (4)..(4): Xaa is norleucine
SXDXRCSASVECGQKCLKAIGSIFGKCMNKKCKCYPR

SEQ ID NO: 73 - protein, 37 aa, Artificial Sequence
  Modified SEQ ID NO 1
  MISC_FEATURE (2)..(2): Xaa is norleucine
  MISC_FEATURE (4)..(4): Xaa is norleucine
SXDXRCSASVECVQKCLKAIGSIFGKCMNKKCKCYPR

SEQ ID NO: 74 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (14)..(14): Xaa is 2-amino-5-carboxypentanoyl
SXDXRCSASVECKXKCLKAIGSIFGKCMNKKCKCYPR

SEQ ID NO: 75 - protein, 36 aa, Artificial Sequence
  Modified SEQ ID NO 1
  MISC_FEATURE (2)..(2): Xaa is norleucine
  MISC_FEATURE (4)..(4): Xaa is norleucine
SXDXRCSASVECKQKCLKAIGSIFGKCMNKKCKCYQ

SEQ ID NO: 76 - protein, 37 aa, Artificial Sequence
  Modified SEQ ID NO 1
  MISC_FEATURE (2)..(2): Xaa is norleucine
  MISC_FEATURE (4)..(4): Xaa is norleucine
SXDXRCSASVECKQKCLKAIGSIFGKCMNKKCRCYPR

SEQ ID NO: 77 - protein, 37 aa, Artificial Sequence
  Modified SEQ ID NO 1
  MISC_FEATURE (2)..(2)
SXDERCSASVECKQKCLKAIGSIFGKCMNKKCKCYPR

SEQ ID NO: 78 - protein, 37 aa, Artificial Sequence
  Modified SEQ ID NO 1
  MISC_FEATURE (2)..(2): Xaa is norleucine
  MISC_FEATURE (4)..(4): Xaa is norleucine
SXDXRCSASVECKQKCLVAIGSIFGKCMNKKCKCYPR

SEQ ID NO: 79 - protein, 37 aa, Artificial Sequence
  Modified SEQ ID NO 1
  MISC_FEATURE (2)..(2): Xaa is norleucine
  MISC_FEATURE (4)..(4): Xaa is norleucine
SXDXRCSASVECAQSCLKAIGSIFGKCMNKKCKCYPR

SEQ ID NO: 80 - protein, 37 aa, Artificial Sequence
  Modified SEQ ID NO 1
  MISC_FEATURE (2)..(2): Xaa is norleucine
  MISC_FEATURE (4)..(4): Xaa is norleucine
SXDXRCSASVECAQKCLAAIGSIFGKCMNKKCKCYPR

SEQ ID NO: 81 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXDXRCSASVECKQKCLKAIGCIFGKCXNKKCKCYPC

SEQ ID NO: 82 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
SXDXRCSASVECKEKCLQAIGSIFGKCXNKKCKCYPR

SEQ ID NO: 83 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (18)..(18): Gln is homo-glutamine
  MISC_FEATURE (28)..(28): Xaa is norleucine
PXDXRCSASVECKEKCLEAIGSIFGKCXNKKCKCYPR

SEQ ID NO: 84 - protein, 32 aa, Artificial Sequence
  Modified SEQ ID NO 1
  MISC_FEATURE (23)..(23): Xaa is norleucine
CSASVECKQKCLKAIGCIFGKCXNKKCKCYPC

SEQ ID NO: 85 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
SXDXRCSASVECKQKCLAAIGCIFGKCXNKKCKCYPC

SEQ ID NO: 86 - protein, 37 aa, Artificial Sequence; identical to SEQ ID NO: 71
  Modified SEQ ID NO 1
  MISC_FEATURE (2)..(2): Xaa is norleucine
  MISC_FEATURE (4)..(4): Xaa is norleucine
  MISC_FEATURE (35)..(35): Xaa is 4-fluoro-phenylalanine

SEQ ID NO: 87 - protein, 37 aa, Artificial Sequence; identical to SEQ ID NO: 71
  Modified SEQ ID NO 1
  MISC_FEATURE (2)..(2): Xaa is norleucine
  MISC_FEATURE (4)..(4): Xaa is norleucine
  MISC_FEATURE (35)..(35): Xaa is 4-nitro-phenylalanine

SEQ ID NO: 88 - protein, 37 aa, Artificial Sequence; identical to SEQ ID NO: 71
  Modified SEQ ID NO 1
  MISC_FEATURE (2)..(2): Xaa is norleucine
  MISC_FEATURE (4)..(4): Xaa is norleucine
  MISC_FEATURE (35)..(35): Xaa is 4-methyl-phenylalanine

SEQ ID NO: 89 - protein, 36 aa, Artificial Sequence
  Modified SEQ ID NO 1
  MISC_FEATURE (1)..(1): Xaa is norleucine
  MISC_FEATURE (3)..(3): Xaa is norleucine
  MISC_FEATURE (27)..(27): Xaa is norleucine
XDXRCSASVECKQKCLKAIGSIFGKCXNKKCKCYPR

SEQ ID NO: 90 - protein, 37 aa, Artificial Sequence
  Modified SEQ ID NO 1
  MISC_FEATURE (4)..(4): Xaa is norleucine
  MISC_FEATURE (28)..(28): Xaa is norleucine
NIDXRCSASVECKQKCLKAIGSIFGKCXNKKCKCYPR

SEQ ID NO: 91 - protein, 39 aa, Artificial Sequence
  Modified SEQ ID NO 1
  MISC_FEATURE (4)..(4): Xaa is norleucine
PIEXRCSASVECKQKCLKAIGSIFGKCNLENKKCKCYPR

SEQ ID NO: 92 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXEXRCSASVECKQKCLLAIGSIFGKCXNKKCKCYPR

SEQ ID NO: 93 - protein, 37 aa, Artificial Sequence
  Modified SEQ ID NO 1
  MISC_FEATURE (28)..(28): Xaa is norleucine
PIDERCSASVECKQKCLAAIGSIFGKCXNKKCKCYPR

SEQ ID NO: 94 - protein, 37 aa, Artificial Sequence
  Modified SEQ ID NO 1
  MISC_FEATURE (4)..(4): Xaa is norleucine
  MISC_FEATURE (28)..(28): Xaa is norleucine
PIEXRCSASVECKQKCLAAIGSIFGKCXNKKCKCYPR

SEQ ID NO: 95 - protein, 37 aa, Artificial Sequence
  Modified SEQ ID NO 1
  MISC_FEATURE (2)..(2): Xaa is norleucine
  MISC_FEATURE (4)..(4): Xaa is norleucine
PXDXRCSASVECAQKCLAAIGSIFGKCMNKKCKCYPR

SEQ ID NO: 96 - protein, 37 aa, Artificial Sequence
  Modified SEQ ID NO 1
  MISC_FEATURE (2)..(2): Xaa is norleucine
  MISC_FEATURE (4)..(4): Xaa is norleucine
  MISC_FEATURE (28)..(28): Xaa is norleucine
PXEXRCSASVECAQKCLAAIGSIFGKCXNKKCKCYPR

SEQ ID NO: 97 - protein, 32 aa, Artificial Sequence
  Modified SEQ ID NO 1
  MISC_FEATURE (23)..(23): Xaa is norleucine
CSASVECKQKCLKAIGSIFGKCXNKKCKCYPR

SEQ ID NO: 98 - protein, 33 aa, Artificial Sequence
  Modified SEQ ID NO 1
  MISC_FEATURE (24)..(24): Xaa is norleucine
RCSASVECKQKCLKAIGSIFGKCXNKKCKCYPR

SEQ ID NO: 99 - protein, 34 aa, Artificial Sequence
  Modified SEQ ID NO 1
  MISC_FEATURE (25)..(25): Xaa is norleucine
SKCSASVECKQKCLKAIGSIFGKCXNKKCKCYPR

SEQ ID NO: 100 - protein, 34 aa, Artificial Sequence
  Modified SEQ ID NO 1
  MISC_FEATURE (25)..(25): Xaa is norleucine
LRCSASVECKQKCLKAIGSIFGKCXNKKCKCYPR

SEQ ID NO: 101 - protein, 34 aa, Artificial Sequence
  Modified SEQ ID NO 1
  MISC_FEATURE (25)..(25): Xaa is norleucine
LRCSASVECKQKCLAAIGSIFGKCXNKKCKCYPR

SEQ ID NO: 102 - protein, 34 aa, Artificial Sequence
  Modified SEQ ID NO 1
LRCSASVECKQKCLAAIGSIFGKCMNKKCKCYPR

SEQ ID NO: 103 - protein, 32 aa, Artificial Sequence
  Modified SEQ ID NO 1
CSASVECKQKCLKAIGSIFGKCMNKKCKCYPR

SEQ ID NO: 104 - protein, 35 aa, Artificial Sequence
  ShK
RSCIDTIPKSRCTAFQCKHSMKYRLSFCRKTCGTC

SEQ ID NO: 105 - protein, 37 aa, Artificial Sequence
  ShK-186
  MISC_FEATURE (1)..(1): Xaa is phosphotyrosyl
  MISC_FEATURE (2)..(2): Xaa is 8-Amino-3,6-dioxoaoctanoyl
XXRSCIDTIPKSRCTAFQCKHSMKYRLSFCRKTCGTC

SEQ ID NO: 106 - protein, 34 aa, Artificial Sequence
  Moka1
INVKCSLPQQCIKPCKDAGMRFGKCMNKKCRCYS

SEQ ID NO: 107 - protein, 36 aa, Artificial Sequence
  Vm24
AAAISCVGSPECPPKCRAQGCKNGKCMNRKCKCYYC

SEQ ID NO: 108 - protein, 5 aa, Artificial Sequence
  Positions 1-5 sequence embodiment
NMDMR

SEQ ID NO: 109 - protein, 5 aa, Artificial Sequence
  Positions 1-5 sequence embodiment
  MISC_FEATURE (2)..(2): Xaa is norleucine
  MISC_FEATURE (4)..(4): Xaa is norleucine
NXDXR

SEQ ID NO: 110 - protein, 5 aa, Artificial Sequence
  Positions 1-5 sequence embodiment
  MISC_FEATURE (2)..(2): Xaa is norleucine
  MISC_FEATURE (4)..(4): Xaa is norleucine
NXEXR

SEQ ID NO: 111 - protein, 5 aa, Artificial Sequence
  Positions 1-5 sequence embodiment
  MISC_FEATURE (2)..(2): Xaa is norleucine
  MISC_FEATURE (4)..(4): Xaa is norleucine
PXEXR

(19) United States
(12) Patent Application Publication
(10) Pub. No.: US 2024/0182531 A1
(43) Pub. Date: Jun. 6, 2024

(54) KV1.3 Blockers

(71) Applicant: Zealand Pharma A/S, Søborg (DK)

(72) Inventor: Henrik Fischer Munch, Søborg (DK)

(21) Appl. No.: 18/282,566

(22) PCT Filed: Mar. 22, 2022
(86) PCT No.: PCT/EP2022/057533

§ 371 Date: Sep. 18, 2023

(30) Foreign Application Priority Data
Mar. 23, 2021 (EP) 21164384.6
Dec. 9, 2021 (EP) 21213431.6

(51) Int. Cl. C07K 14/435; A61K 38/00; A61P 29/00

(52) U.S. Cl. CPC C07K 14/43522; A61P 29/00; A61K 38/00

(57) ABSTRACT

The present invention provides novel blockers of the potassium channel Kv1.3, polynucleotides encoding them, and methods of making and using them.

### FIELD OF THE INVENTION

The present invention provides novel blockers of the potassium channel Kv1.3, polynucleotides encoding them, and methods of making and using them.

### BACKGROUND TO THE INVENTION

Ion channels are membrane proteins which form pores in biological membranes, permitting (and regulating) the flow of ions across the relevant membrane. There are numerous different types of ion channel, which may be classified in various ways, such as by the species of ions to which they provide passage, the way in which passage of ions is regulated or “gated” (e.g. “ligand-gated” or “voltage-gated”), and their cellular or sub-cellular localisation.

Potassium channels fall into four major classes, namely voltage-gated potassium channels, calcium-activated potassium channels, inwardly rectifying potassium channels, and tandem pore domain potassium channels.

The voltage-gated potassium channels, like other voltage gated channels, open or close in response to transmembrane voltages. They represent a complex family with diverse biological functions, including the regulation of neurotransmitter release, heart rate, insulin secretion, neuronal excitability, epithelial electrolyte transport, smooth muscle contraction, and cell volume.

The Kv1.3 (potassium voltage-gated channel subfamily A member 3) channel is expressed on T cells and plays a role in regulating T cell activation. Blockers of Kv1.3 have been shown to inhibit proliferation of activated T cells in vitro (reviewed in Cahalan and Chandy, Immunol. Rev. 231:59-87, 2009), and to inhibit T cell-dependent disease progression in various experimental models of autoimmune disease including experimental autoimmune encephalomyelitis (EAE), experimental arthritis, delayed-type hypersensitivity (DTH), allergic contact dermatitis and glomerulonephritis. See, for example, Rangaraju et al. (Expert Opin. Ther. Targets 13:909-24, 2009); Beeton et al. (Proc. Natl. Acad. Sci. USA. 103:17414-9, 2006); Koo et al. (J. Immunol. 158:5120-8, 1997); Hyodo et al. (Am. J. Physiol. Renal Physiol. 299: F1258-69, 2010). WO 2016/112208 describes topical application of Kv1.3 blockers for the treatment of skin and mucosal inflammation.

Thus, Kv1.3 blockers have considerable potential for use in treatment of inflammatory disorders, including autoimmune disorders.

WO 2015/013330 proposes use of Kv1.3 blocker peptides for treatment of ophthalmic conditions, such as dry eye and uveitis, including when caused by autoimmune conditions such as Sjogren's syndrome.

Blockers of Kv1.3 may also have beneficial metabolic effects, e.g. in relation to energy homeostasis, body weight regulation, and glucose control. Kv1.3 knock-out (Kv1.3(−/−)) mice exhibit reduced weight gain, higher insulin sensitivity, and reduced plasma glucose levels in response to a high fat diet as compared to control littermates (Xu et al., Hum. Mol. Genet. 12:551-9, 2003). Further, Kv1.3 blockers have been shown to increase expression in skeletal muscle and adipose tissue of glucose transporter 4 (GLUT4), to increase insulin sensitivity in normal and ob/ob obese mice, and to increase glucose uptake in primary adipocytes in vitro (Xu et al., Proc. Natl. Acad. Sci. USA 101:3112-7, 2004). In humans, a single nucleotide polymorphism (SNP) in the Kv1.3 gene has also been associated with decreased insulin sensitivity and impaired glucose tolerance (Tschritter, Clin Endocrinol Metab 91: 654-8, 2006).

Kv1.3 is also expressed in proliferating human and mouse smooth muscle cells. Blockers of Kv1.3 may be effective in smooth muscle proliferative disorders such as restenosis, e.g. in patients following vascular surgery (e.g. angioplasty). Kv1.3 blockers have been shown to inhibit calcium entry, reduce smooth muscle cell migration, and inhibit neointimal hyperplasia in ex vivo human vein samples (Cheong et al., Cardiovasc. Res. 89:282-9, 2011).

Further evidence suggests that Kv1.3 channels are involved in the activation and/or proliferation of many types of cells, including tumor cells (Bielanska et al., Curr. Cancer Drug Targets 9:904-14, 2009), microglia (Khanna et al., Am. J. Physiol. Cell Physiol. 280: C796-806, 2001) and differentiation of neuronal progenitor cells (Wang et al., J. Neurosci. 30:5020-7, 2010). Kv1.3 blockers may therefore be beneficial in the treatment of neuroinflammatory and neurodegenerative disorders, and cancers.

Kv1.3 is part of a sub-family of closely related potassium channels, designated Kv1.1 to Kv1.8. When dealing with large homologous families, it is always desirable for a blocker to be as selective and specific as possible for the desired target, to improve efficacy and safety, and avoid undesirable off-target effects. The most specific Kv1.3 blockers identified to date are venom peptides derived from various types of venomous organisms, such as snakes, arachnids (such as scorpions and spiders), sea anemones, etc. Such Kv1.3 blockers include the peptides ShK, Oskl, margatoxin and kaliotoxin, reviewed by Chandy et al., Trends in Pharmacol. Sci. 25:280-9, 2004. See also Abdel-Mottaleb et al., Toxicon 51:1424-30, 2008, and Mouhat et al., Biochem. J. 385(Pt 1): 95-104, 2005.

Various attempts to engineer toxin peptides for particular properties, including specificity or potency, have been described, e.g. in WO2006/002850, WO2006/042151, WO2008/088422, WO2006/116156, WO2010/105184 and WO2014/116937. International patent application PCT/EP2020/076187 also disclosed Kv1.3 blockers.

However, there remains a need for alternative Kv1.3 blockers. Blockers having improved specificity compared to known blockers may be particularly desirable, although improvements in other properties such as stability and potency may also be useful.

### SUMMARY OF THE INVENTION

The invention relates to ion channel blockers derived from a toxin peptide of the scorpion Parabuthus transvaalicus. The toxin peptide has the amino acid sequence

| (SEQ ID NO: 1) | |
|---|---|
| | QMDMRCSASVECKQKCLKAIGSIFGKCMNKKCKCYPR. |

Amongst other desirable properties, this molecule and derivatives or variants thereof have been found to be extremely selective blockers for the Kv1.3 potassium ion channel over other voltage-gated potassium channels, and typically also have high potency at blocking the Kv1.3 channel.

Accordingly, the invention provides an ion channel blocker which has Kv1.3 inhibitor activity, or a pharmaceutically acceptable salt thereof, comprising a variant of the sequence QMDMRCSASVECKQKCLKAIGSIFGKCMNKKCKCYPR (SEQ ID NO: 1), wherein at least one amino acid in position 7, 8, 9, 10 or 11 of SEQ ID NO: 1 is substituted with an amino acid having a positively charged side chain and/or an amino acid having an aromatic side chain, and wherein the variant does not comprise any of the following sequences:

SEQ ID NO: 2
NMDMRCKASVECKQKCLKAIGSIFGKCMNKKCKCYPR
SEQ ID NO: 3
NMDMRCSASRECKQKCLKAIGSIFGKCMNKKCKCYPR
SEQ ID NO: 4
N[Nle]D[Nle]RCRASVECKQKCLKAIGSIFGKC[Nle]NKKCKCYPR
SEQ ID NO: 5
N[Nle]D[Nle]RCSHSVECKQKCLKAIGSIFGKC[Nle]NKKCKCYPR
SEQ ID NO: 6
N[Nle]D[Nle]RCSASKECKQKCLKAIGSIFGKC[Nle]NKKCKCYPR
SEQ ID NO: 7
P[Nle]E[Nle]RCFASVECKQKCLAAIGSIFGKC[Nle]NKKCKCYPR
SEQ ID NO: 8
P[Nle]E[Nle]RCSYSVECKQKCLAAIGSIFGKC[Nle]NKKCKCYPR
SEQ ID NO: 9
P[Nle]E[Nle]RCSAFVECKQKCLAAIGSIFGKC[Nle]NKKCKCYPR

In one aspect the invention provides an ion channel blocker which has Kv1.3 inhibitor activity, or a pharmaceutically acceptable salt thereof, comprising a variant of the sequence QMDMRCSASVECKQKCLKAIGSIFGKCMNKKCKCYPR (SEQ ID NO: 1), wherein at least one amino acid in position 7, 8, 9, 10 or 11 of SEQ ID NO: 1 is substituted with an amino acid having a positively charged side chain and/or an amino acid having an aromatic side chain.

In other aspects the invention provides a nucleic acid encoding an ion channel blocker of the invention, an expression vector comprising a nucleic acid of the invention, and a host cell comprising a nucleic acid of the invention or an expression vector of the invention and capable of expressing an ion channel blocker of the invention.

The invention also provides methods of synthesising the ion channel blocker or pharmaceutically acceptable salt of the invention.

The invention further provides pharmaceutical compositions comprising the ion channel blocker or pharmaceutically acceptable salt of the invention.

The invention yet further provides medical use of the ion channel blocker, pharmaceutically acceptable salt or pharmaceutical composition of the invention and methods of treating disease using the ion channel blocker, pharmaceutically acceptable salt or pharmaceutical composition of the invention.

### DETAILED DESCRIPTION

### Ion Channel Blocker

The invention provides an ion channel blocker.

Ion channel blockers of the invention may be in the form of a pharmaceutically acceptable salt. All references to “an ion channel blocker” herein should be considered to encompass any pharmaceutically acceptable salt thereof, regardless of whether “pharmaceutically acceptable salt” is explicitly recited.

The term “ion channel blocker” is used to denote a compound having inhibitor (or blocking) activity against an ion channel, i.e. capable of inhibiting or eliminating ion flow through the respective ion channel, typically by binding to the ion channel. Similarly, the terms“Kv1.3 inhibitor” and “Kv1.3 inhibitor component” refer to a peptide capable of inhibiting or eliminating ion flow through a Kv1.3 ion channel, typically by binding to the Kv1.3 channel. However, the terms “blocker” and “inhibitor” should not be taken to imply any particular mechanism of action, or any particular mode of interaction with the ion channel itself.

The term Kv1.3 refers to potassium voltage-gated channel subfamily A member 3, also referred to as KCNA3, HPCN3, HGK5, HuKIII and HLK3. “Subfamily A” may also be referred to as “shaker-related subfamily”. The human amino acid sequence is provided under UniProt accession number P22001, version P22001.3 (Q5VWN2).

The Kv1.3 channel is expressed on T and B lymphocytes and has been implicated in T cell activation. A number of groups are pursuing development of Kv1.3 blockers for the inhibition of immune responses as well as for various other indications. However, the Kv1.3 channel is part of a complex family of related ion channels, also including the Kv1.1, Kv1.2 and Kv1.6 channels, which have different physiological roles. Consequently it is desirable for Kv1.3 inhibitors to be as selective as possible for Kv1.3 in preference to other ion channels, especially other voltage-gated potassium channels, such as Kv1.1, Kv1.2, Kv1.4, Kv1.5, Kv1.6, Kv1.7 and Kv1.8.

The ion channel blocker or pharmaceutically acceptable salt of the invention has Kv1.3 inhibitor activity. In other words, the ion channel blocker of the invention (and the peptide component of the ion channel blocker in isolation) has inhibitor or blocker activity at the Kv1.3 ion channel, i.e. it is capable of inhibiting ion flow through the Kv1.3 channel.

### IC₅₀ Values

IC₅₀ values may be used as a measure of inhibitor (or blocker) activity or potency. An IC₅₀ value is a measure of the concentration of an inhibitor required to achieve half of that compound's maximal inhibition of ion channel activity in a given assay. A compound which has a lower IC₅₀ at a particular ion channel than a reference compound can be considered to be a more active inhibitor, or a more potent inhibitor, than the reference compound. The terms “activity” and “potency” are used interchangeably.

IC₅₀ values may be determined using any appropriate assay, such as fluorescence-based assays measuring ion flux (e.g. thallium ion flux) and patch clamp assays. They may be performed as described in the Examples below. Patch clamp assays may be preferred, e.g. using the QPatch® system.

In some embodiments, the ion channel blocker of the invention has an IC₅₀ for human Kv1.3 potassium channel of 50 nM or less, such as 20 nM or less, such as 10 nM or less, such as 5 nM or less, such as 2 nM or less, such as 1 nM or less, such as 0.5 nM or less, such as 0.4 nM or less, such as 0.3 nM or less, such as 0.2 nM or less. Preferably, the ion channel blocker of the invention has an IC₅₀ for human Kv1.3 potassium channel of 0.5 nM or less, most preferably 0.2 nM or less.

Ion channel blockers of the invention include compounds 1-12 as described herein, which are shown in Example 2 herein to have an IC₅₀ of 50 nM or less for human Kv1.3 potassium channel. Ion channel blockers of the invention include compounds 1-5 and 7-12 as described herein, which are shown in Example 2 herein to have an IC₅₀ of 5 nM or less for human Kv1.3 potassium channel. Ion channel blockers of the invention include compounds 1-5, 7, 8 and 10-12 as described herein, which are shown in Example 2 herein to have an IC₅₀ of 0.5 nM or less for human Kv1.3 potassium channel. Ion channel blockers of the invention include compounds 1-5, 7, 8 and 11 as described herein, which are shown in Example 2 herein to have an IC₅₀ of 0.3 nM or less for human Kv1.3 potassium channel. Ion channel blockers of the invention include compounds 1-5 as described herein, which are shown in Example 2 herein to have an IC₅₀ of 0.2 nM or less for human Kv1.3 potassium channel.

### Half-Life

In some embodiments, the ion channel blocker of the invention has an in vivo half-life of at least 1 hour, such as at least 1.5 hours, such as at least 2 hours, such as at least 2.5 hours.

Half-life (T_{1/2}) of an ion channel blocker may be determined using assays known in the art, such as the described in Example 3 herein.

### Selectivity

The ion channel blockers of the invention are selective for Kv1.3. In an embodiment the ion channel blockers of the invention are selective over Kv1.1, Kv1.2, Kv1.4, Kv1.5, Kv1.6, Kv1.7 and Kv1.8. In particular, the ion channel blockers of the invention are selective for Kv1.3 over one or more of Kv1.1, Kv1.2 and Kv1.6.

For example, they may be selective for Kv1.3 over Kv1.1; selective for Kv1.3 over Kv1.2; selective for Kv1.3 over Kv1.6; selective for Kv1.3 over Kv1.1 and Kv1.2; selective for Kv1.3 over Kv1.1 and Kv1.6; selective for Kv1.3 over Kv1.2 and Kv1.6; or selective for Kv1.3 over Kv1.1, Kv1.2 and Kv1.6. Typically, the ion channel blockers are selective for Kv1.3 over Kv1.1. They may additionally be selective for Kv1.3 over Kv1.2 and/or Kv1.6.

“Selective” in this context means that the ion channel blockers have higher inhibitor activity against Kv1.3 than against the respective ones of Kv1.1, Kv1.2 and Kv1.6. Thus, their IC₅₀ against Kv1.3 is typically lower than against the respective other ion channel or channels.

Selectivity for Kv1.3 over another ion channel X may therefore be expressed as a ratio of the respective IC₅₀ values, e.g. as IC₅₀[X]/IC₅₀[Kv1.3].

The ion channel blockers of the invention may therefore have a selectivity for Kv1.3 over Kv1.1 of at least 10, at least 100, at least 1000, or at least 10000, and may be up to 100000 or even higher. Typically, the ion channel blockers of the invention have a selectivity for Kv1.3 over Kv1.1 of at least 100, or at least 1000.

The ion channel blockers of the invention may therefore have a selectivity for Kv1.3 over Kv1.2 of at least 10, at least 100, at least 1000, or at least 10000, and may be up to 100000 or even higher. Typically, they have a selectivity for Kv1.3 over Kv1.2 of at least 10, and preferably at least 50 or at least 100 or at least 1000.

The ion channel blockers of the invention may therefore have a selectivity for Kv1.3 over Kv1.6 of at least 10, at least 100, at least 1000, or at least 10000, and may be up to 100000 or even higher. Typically, they have a selectivity for Kv1.3 over Kv1.6 of at least 100, or at least 400, or at least 1000.

The ion channel blockers of the invention may have greater selectivity than known ion channel blockers such as ShK, Mokatoxin (Moka1), Vm24, Odk2 or Osk1. Thus the ion channel blockers of the invention may have higher selectivity for Kv1.3 over ion channel X, i.e. IC₅₀[X]/IC₅₀[Kv1.3], which is greater than the selectivity of the comparison molecule. The selectivity of the two ion channel blockers will be determined under the same conditions for each ion channel to enable direct comparison. As mentioned above, any appropriate assays may be used, such as fluorescence-based ion flux assays and patch clamp assays.

The ion channel blockers of the invention may have lower absolute inhibitor activity (i.e. higher IC₅₀) than known ion channel blockers (such as Odk2 or Osk1) at any or all of Kv1.1, Kv1.2 and/or Kv1.6. However, it may be acceptable for them to have higher absolute inhibitor activity at any or all of these ion channels, as long as their selectivity for Kv1.3 is higher than that of the comparison compound. Typically, though, the compounds of the invention combine high specificity for Kv1.3 with high potency.

### Peptide

The ion channel blocker of the invention comprises a variant of the peptide sequence

| (SEQ ID NO: 1) | |
|---|---|
| | QMDMRCSASVECKQKCLKAIGSIFGKCMNKKCKCYPR. |

SEQ ID NO: 1 is the amino acid sequence of a toxin peptide of the scorpion Parabuthus transvaalicus. As described herein, this peptide is a selective Kv1.3 potassium ion channel inhibitor.

### Amino Acids

Throughout the present description and claims the conventional three-letter and one-letter codes for naturally occurring amino acids are used, i.e. A (Ala), G (Gly), L (Leu), I (Ile), V (Val), F (Phe), W (Trp), S (Ser), T (Thr), Y (Tyr), N (Asn), Q (Gln), D (Asp), E (Glu), K (Lys), R (Arg), H (His), M (Met), C (Cys) and P (Pro);

as well as generally accepted codes for other α-amino acids, such as sarcosine (Sar), norleucine (Nle), α-aminoisobutyric acid (Aib), 2,3-diaminopropanoic acid (Dap), 2,4-diaminobutanoic acid (Dab), 2,5-diaminopentanoic acid (ornithine or Orn), alpha-aminobutyric acid (Abu, also known as homo-alanine), hK (also known as hLys or homo-Lys (homo-lysine)), hQ (also known as hGln or homo-Gln (homo-glutamine, also known as 6-oxolysine)), L-5-carbamoylnorvaline, 6-amino-6-oxonorleucine, 5-(aminocarbonyl)norvaline), F(4-F) (4-fluoro-phenylalanine), F(4-NH₂) (4-amino-phenylalanine), F(4-NO₂) (4-nitro-phenylalanine), and F(4-CH₃) (4-methyl-phenylalanine).

The designation [2-Amino-5-carboxypentanoyl] indicates a peptide residue of 2-amino-5-carboxypentanoic acid, which has a side chain similar to that of glutamic acid, but with an additional methylene group, as shown in the following structure:

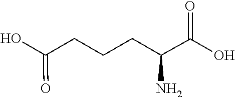 [SMILES: N[C@@H](CCCC(=O)O)C(=O)O]

The designation [2,3-Diaminopropanoyl] indicates a peptide residue of 2,3-Diaminopropanoic acid, which has the following structure:

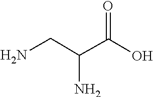

The designation [2,4-Diaminobutanoyl] indicates a peptide residue of 2,4-Diaminobutanoic acid, which has the following structure:

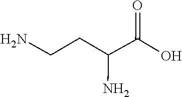

The designation [2-Amino-3-guanidinopropionyl] indicates a peptide residue of 2-Amino-3-guanidinopropionic acid, which has the following structure:

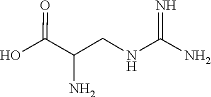

Such other α-amino acids may be shown in square brackets “[ ]” (e.g. “[Nle]”) when used in a general formula or sequence in the present specification, especially when the rest of the formula or sequence is shown using the single letter code. Unless otherwise specified, amino acid residues in peptides of the invention are of the L-configuration. However, D-configuration amino acids may be incorporated. In the present context, an amino acid code written with a small letter represents the D-configuration of said amino acid, e.g. “k” represents the D-configuration of lysine (K).

The variant of SEQ ID NO: 1 contains six cysteine (C) residues which together form three disulphide bonds, between residues 6C and 27C, residues 12C and 32C, and residues 16C and 34C.

The disulphide bonds may be indicated graphically as follows by reference to SEQ ID NO: 1:
QMDMRC(1)SASVEC(2)KQKC(3)LKAIGSIFGKC(1)MNKKC(2)KC(3)YPR
   where a pair of cysteine residues which participate together in a disulphide bond are indicated by the same numeral in parentheses. Similar notation can be applied to any of the other sequences in this application. Except where the context demands otherwise, it should be understood that an active inhibitor compound includes appropriate disulphide bonding.

It may be desirable that no other cysteine residues are introduced into the variant of SEQ ID NO: 1 by substitution or insertion. Thus, in some embodiments, the variant contains no other cysteine residues apart from those at positions corresponding to positions 6, 12, 16, 27, 32 and 34 of SEQ ID NO: 1. In some embodiments, any substitutions or deletions in the variant of SEQ ID NO: 1 are not at amino acid positions 6, 12, 16, 27, 32 and 34 of SEQ ID NO: 1.

### Variants

The ion channel blocker of the invention comprises a variant of SEQ ID NO: 1. In some embodiments, the ion channel blocker of the invention consists of a variant of SEQ ID NO: 1. Variants of SEQ ID NO: 1 are peptides comprising one or more amino acid that is different to those of SEQ ID NO: 1 (i.e. one or more amino acid changes compared to SEQ ID NO: 1). Such variants may also be termed “derivatives”, “variant peptides”, “peptides” or “compounds”. Variants of SEQ ID NO: 1 may differ from SEQ ID NO: 1 in that one or more amino acid of SEQ ID NO: 1 is deleted and/or one or more amino acid of SEQ ID NO: 1 is substituted for a different amino acid and/or one or more amino acid is inserted into the sequence of SEQ ID NO: 1. Amino acids may be inserted at an internal position of SEQ ID NO: 1, at the N-terminus of SEQ ID NO: 1 or at the C-terminus of SEQ ID NO: 1. Thus the variants of SEQ ID NO: 1 comprises one or more substitutions, insertions and/or deletions.

Except where otherwise noted, a “substitution” refers to the substitution (i.e. replacement) of a single amino acid in SEQ ID NO: 1. Thus, for example, substitution of three contiguous amino acids in SEQ ID NO: 1 constitutes three substitutions, rather than a single substitution. Likewise, “insertion” refers to insertion of a single amino acid into SEQ ID NO: 1 (which may be internal, at the N-terminus, and/or at the C-terminus), so insertion of, for example, three contiguous amino acids in SEQ ID NO: 1 constitutes three insertions, rather than a single insertion. “Deletion” refers to deletion of a single amino acid from SEQ ID NO: 1, so deletion of, for example, three contiguous amino acids in SEQ ID NO: 1 constitutes three deletions, rather than a single deletion.

In particularly preferred embodiments, the variant of SEQ ID NO: 1 differs from SEQ ID NO: 1 by up to ten substitutions, insertions or deletions in total. In some such embodiments, the variant contains 10, 9, 8, 7, 6, 5, 4, 3, 2 or 1 substitutions, insertions or deletions in total compared to SEQ ID NO: 1. Preferably, the variant differs from SEQ ID NO: 1 by 7 substitutions, insertions or deletions in total compared to the sequence of SEQ ID NO: 1.

### Numbering

The amino acid residues of SEQ ID NO: 1 are numbered from 1 to 37, in the conventional direction of N- to C-terminus. Throughout this specification, amino acid positions in variants of SEQ ID NO: 1 are numbered according to the corresponding position in SEQ ID NO: 1 when optimally aligned therewith. Thus, especially for variants which contain one or more insertions or deletions compared to SEQ ID NO: 1, the numbering of any given residue reflects the corresponding residue in SEQ ID NO: 1 and does not necessarily reflect its linear position in the sequence of the variant.

The residue present at a specific position may be indicated by the number of the relevant position alongside the single letter code or three letter code for the residue present. Thus, 1Q or Q1 (the two formats are interchangeable) indicates a glutamine (Q) residue at position 1, while 2Nle, 2[Nle], Nle2 or [Nle]2 indicates a norleucine residue at position 2.

An asterisk may be used to denote the position of a deletion relative to the sequence of SEQ ID NO: 1. For example, “1*” indicates a deletion of the residue at position 1 as compared to SEQ ID NO: 1.

An insertion may be indicated by a string of consecutive residues at a single position, e.g. “1QA” indicates an insertion of an alanine (A) residue after the glutamine (Q) residue at position 1.

In some embodiments, any substitutions compared to SEQ ID NO: 1 are conservative substitutions. However, any substitution listed in any of the generic formulae provided below may be introduced at the respective position.

In some embodiments, any substitutions or deletions in the variant of SEQ ID NO: 1 are at amino acid positions selected from positions 1-5, 7-11, 13-15, 17-23, 25, 28-31, 33 and 35-37 of SEQ ID NO: 1. Preferably, any substitutions or deletions in the variant of SEQ ID NO: 1 are at amino acid positions selected from positions 1-5, 7-11, 13, 15, 18, 28 and 30 of SEQ ID NO: 1.

### Terminal Groups

A “H” (or “Hy-”) moiety at the N-terminus of a sequence indicates a hydrogen atom [i.e. R¹=hydrogen], corresponding to the presence of a free primary or secondary amino group at the N-terminus. Alternatively, a variant peptide may comprise an alternative N-terminal group (i.e. an N-terminal modification).

Thus, in some embodiments of the ion channel blocker or pharmaceutically acceptable salt of the invention, the peptide comprises at the N-terminus a group selected from C₁₋₄ alkyl, acetyl (Ac), formyl, benzoyl and trifluoroacetyl.

An “—OH” moiety at the C-terminus of the sequence indicates the presence of a hydroxy group (OH) as part of a carboxy (COOH) group at the C-terminus of the molecule. An “—NH₂” moiety at the C-terminus of the sequence indicates the presence of an amino group (NH₂) as part of an amido (CONH₂) group at the C-terminus of the molecule. A “CH₂OH” moiety at the C-terminus indicates the presence of a hydroxyl group linked to an alkyl group at the C-terminus of the molecule.

Thus, in some embodiments of the ion channel blocker or pharmaceutically acceptable salt of the invention, the peptide comprises at the C-terminus an amino group (—NH₂), a hydroxyl group (—OH) or a hydroxymethyl group (—CH₂OH), preferably an amino group (—NH₂) or a hydroxyl group (—OH).

### Sequence Identity

In some embodiments, the variant has at least 60% sequence identity with SEQ ID NO: 1, such as at least 65% sequence identity, such as at least 70% sequence identity, such as at least 75% sequence identity, such as at least 80% sequence identity, such as at least 85% identity, such as at least 90% sequence identity, such as at least 95% sequence identity, such as at least 96% sequence identity, such as at least 97% sequence identity, such as at least 98% sequence identity, such as at least 99% sequence identity, such as 100% sequence identity.

“Percent (%) amino acid sequence identity” with respect to the peptide sequences herein is defined as the percentage of amino acids in a candidate sequence that are identical to the amino acids in the wild-type toxin peptide sequence SEQ ID NO: 1 after aligning the sequences and introducing gaps, if necessary, to achieve the maximum percent sequence identity, and not considering any conservative substitutions as part of the sequence identity. Sequence alignment can be carried out by the skilled person using techniques well known in the art, for example using publicly available software such as BLAST, BLAST2 or Align software. For examples, see Altschul et al., Methods in Enzymology 266: 460-480 (1996) or Pearson et al., Genomics 46: 24-36, 1997.

The percentage sequence identities used herein in the context of the present invention may be determined using these programs with their default settings. More generally, the skilled worker can readily determine appropriate parameters for determining alignment, including any algorithms needed to achieve maximal alignment over the full length of the sequences being compared.

### Particular Peptide Variants

### Positions 7-11

The ion channel blocker or a pharmaceutically acceptable salt of the invention comprises a variant of the sequence QMDMRCSASVECKQKCLKAIGSIFGKCMNKKCKCYPR (SEQ ID NO: 1) wherein at least one amino acid in position 7, 8, 9, 10 or 11 of SEQ ID NO: 1 is substituted with an amino acid having a positively charged side chain and/or an amino acid having an aromatic side chain.

In some embodiments, at least one amino acid in position 7, 8, 9, 10 or 11 of SEQ ID NO: 1 is substituted with an amino acid having a positively charged side chain. In some embodiments the amino acid having a positively charged side chain is selected from the group consisting of H, K, hK, R, Orn, 2,3-diaminopropanoyl, 2,4-diaminobutanoyl, 2-amino-3-guanidinopropionyl, and 4-amino-phenylalanine (F(4-NH₂)). Preferably the amino acid having a positively charged side chain is selected from H, K, R, Orn, 2,3-diaminopropanoyl, 2-amino-3-guanidinopropionyl and 2,4-diaminobutanoyl. Preferably the amino acid having a positively charged side chain is selected from H, R, Orn, 2,3-diaminopropanoyl, 2-amino-3-guanidinopropionyl and 2,4-diaminobutanoyl.

In some embodiments, at least one amino acid in position 7, 8, 9, 10 or 11 of SEQ ID NO: 1 is substituted with an amino acid having an aromatic side chain. In some embodiments the amino acid having an aromatic side chain is selected from the group consisting of F, Wand Y. Preferably the amino acid having an aromatic side chain is Y.

In some embodiments, the ion channel blocker or a pharmaceutically acceptable salt of the invention comprises a variant of the sequence QMDMRCSASVECKQKCLKAIGSIFGKCMNKKCKCYPR (SEQ ID NO: 1) wherein at least one amino acid in position 7, 9 or 10 of SEQ ID NO: 1 is substituted with an amino acid having a positively charged side chain and/or an amino acid having an aromatic side chain.

In some embodiments, at least one amino acid in position 7, 9 or 10 of SEQ ID NO: 1 is substituted with an amino acid having a positively charged side chain. In some embodiments the amino acid having a positively charged side chain is selected from the group consisting of H, K, hK, R, Orn, 2,3-diaminopropanoyl, 2,4-diaminobutanoyl, 2-amino-3-guanidinopropionyl, and 4-amino-phenylalanine (F(4-NH₂)). Preferably the amino acid having a positively charged side chain is selected from H, K, R, Orn, 2,3-diaminopropanoyl, 2-amino-3-guanidinopropionyl and 2,4-diaminobutanoyl. Preferably the amino acid having a positively charged side chain is selected from H, R, Orn, 2,3-diaminopropanoyl, 2-amino-3-guanidinopropionyl and 2,4-diaminobutanoyl.

In some embodiments, at least one amino acid in position 7, 9 or 10 of SEQ ID NO: 1 is substituted with an amino acid having an aromatic side chain. In some embodiments the amino acid having an aromatic side chain is selected from the group consisting of F, W and Y. Preferably the amino acid having an aromatic side chain is Y.

In some embodiments of the ion channel blocker of the invention, exactly one amino acid in position 7, 8, 9, 10 or 11 of SEQ ID NO: 1 is substituted with an amino acid having a positively charged side chain or an aromatic side chain. In some embodiments of the ion channel blocker of the invention, exactly one amino acid in position 7, 9 or 10 of SEQ ID NO: 1 is substituted with an amino acid having a positively charged side chain or an aromatic side chain.

In some embodiments, the amino acid in position 7 of SEQ ID NO: 1 is substituted with an amino acid having a positively charged side chain or an aromatic side chain. Preferably, the amino acid in position 7 of SEQ ID NO: 1 is substituted with H, K, R, Orn, 2,3-diaminopropanoyl, 2-amino-3-guanidinopropionyl, 2,4-diaminobutanoyl or Y. Preferably, the amino acid in position 7 of SEQ ID NO: 1 is substituted with H, R, Orn, 2,3-diaminopropanoyl, 2-amino-3-guanidinopropionyl, 2,4-diaminobutanoyl or Y.

In some embodiments, the amino acid in position 8 of SEQ ID NO: 1 is substituted with an amino acid having a positively charged side chain or an aromatic side chain. In some embodiments, the amino acid in position 8 of SEQ ID NO: 1 is the same amino acid as position 8 in SEQ ID NO: 1 (i.e. A).

In some embodiments, the amino acid in position 9 of SEQ ID NO: 1 is substituted with an amino acid having a positively charged side chain or an aromatic side chain. Preferably, the amino acid in position 9 of SEQ ID NO: 1 is substituted with K, Orn or 2,3-diaminopropanoyl. Preferably, the amino acid in position 9 of SEQ ID NO: 1 is substituted with Orn or 2,3-diaminopropanoyl.

In some embodiments, the amino acid in position 10 of SEQ ID NO: 1 is substituted with an amino acid having a positively charged side chain or an aromatic side chain. Preferably, the amino acid in position 10 of SEQ ID NO: 1 is substituted with R.

In some embodiments, the amino acid in position 11 of SEQ ID NO: 1 is substituted with an amino acid having a positively charged side chain or an aromatic side chain. Preferably, the amino acid in position 11 of SEQ ID NO: 1 is substituted with R. In some embodiments, the amino acid in position 11 of SEQ ID NO: 1 is the same amino acid as position 11 in SEQ ID NO: 1 (i.e. E).

### Other Positions

In some embodiments, the residue at position 1 is not Q. Glutamine (Q) residues can be unstable, either in vivo or in vitro, e.g. during storage in aqueous solution, which may be of particular relevance when the glutamine residue is located at the N-terminus of the molecule, as the side chain may be sterically capable of interacting with the free alpha amino group, resulting in dehydration to pyroglutamate. Preferably, the amino acid at position 1 is N or P, or is deleted (i.e. the variant of SEQ ID NO: 1 may comprise 1N, 1P or 1*).

In some embodiments, one, two or all three of the amino acids at positions 2, 4 and 28 are not M, since methionine (M) residues are susceptible to oxidation. Preferably one, two or all three methionine amino acids at positions 2, 4 and 28 are either individually substituted with a residue having a non-oxidisable side chain, or are deleted. Any suitable residue with a non-oxidisable residue may be used, with Nle, I, V and L being particularly suitable. Preferably, the amino acids at positions 2, 4 and 28 are all Nle.

In some embodiments of the ion channel blocker of the invention, in the variant of SEQ ID NO: 1,
the amino acid at position 1 is N, P or Q, or is deleted,
the amino acid at position 2 is M or Nle, or is deleted,
the amino acid at position 3 is D or E, or is deleted,
the amino acid at position 4 is M or Nle, or is deleted,
the amino acid at position 5 is R or is deleted,
the amino acid at position 13 is A or K,
the amino acid at position 15 is K or S,
the amino acid at position 18 is A or K,
the amino acid at position 28 is M or Nle, and/or
the amino acid at position 30 is G or K.

In some embodiments of the ion channel blocker of the invention, positions 1-5 of the variant consist of an amino acid sequence selected from NMDMR (SEQ ID NO: 108), N[Nle]D[Nle]R (SEQ ID NO: 109), N[Nle]E[Nle]R (SEQ ID NO: 110), and P[Nle]E[Nle]R (SEQ ID NO: 111), or the amino acids at positions 1-5 are all deleted. Preferably, positions 1-5 of the variant consist of the amino acid sequence P[Nle]E[Nle]R.

In some embodiments of the ion channel blocker of the invention, in the variant of SEQ ID NO: 1,
the amino acids at positions 1-5 consist of the amino acid sequence P[Nle]E[Nle]R,
the amino acid at position 13 is A or K,
the amino acid at position 15 is K or S,
the amino acid at position 18 is A,
the amino acid at position 28 is Nle, and
the amino acid at position 30 is G or K.

In some embodiments of the ion channel blocker of the invention, one or more of the positions 5, 6, 12, 14, 16, 17, 19-27, 29 and 31-37 are the same amino acid as the corresponding position in SEQ ID NO: 1. Preferably, all of positions 5, 6, 12, 14, 16, 17, 19-27, 29 and 31-37 are the same amino acid as the corresponding position in SEQ ID NO: 1. In some embodiments of the ion channel blocker of the invention, one or more of the positions 5, 6, 8, 11, 12, 14, 16, 17, 19-27, 29 and 31-37 are the same amino acid as the corresponding position in SEQ ID NO: 1. Preferably, all of positions 5, 6, 8, 11, 12, 14, 16, 17, 19-27, 29 and 31-37 are the same amino acid as the corresponding position in SEQ ID NO: 1.

### Variants of SEQ ID NO 21

In some embodiments, the ion channel blocker of the invention comprises or consists of a variant of the sequence
P[Nle]E[Nle]RCSASVECKQKCLAAIGSIFGKC[Nle]NKKCKCYPR (SEQ ID NO: 21),
   wherein the variant differs from SEQ ID NO: 21 by 4, 3, 2 or 1 substitutions,
   wherein at least one amino acid in position 7, 8, 9, 10 or 11 of SEQ ID NO: 21 is substituted with an amino acid having a positively charged side chain and/or an amino acid having an aromatic side chain.

SEQ ID NO: 21 is a particular variant of SEQ ID NO: 1. Thus, it will be understood that a variant of SEQ ID NO: 21 is a variant of SEQ ID NO: 1 as described herein.

In other words, in some embodiments, the variant of SEQ ID NO: 1 comprises or consists of a variant of the sequence P[Nle]E[Nle]RCSASVECKQKCLAAIGSIFGKC[Nle]NKKCKCYPR (SEQ ID NO: 21),

wherein the variant differs from SEQ ID NO: 21 by 4, 3, 2 or 1 substitutions,
wherein at least one amino acid in position 7, 8, 9, 10 or 11 of SEQ ID NO: 21 is substituted with an amino acid having a positively charged side chain and/or an amino acid having an aromatic side chain.

Accordingly, all features of embodiments of the variant of SEQ ID NO 1 described herein may apply to the variant of SEQ ID NO: 21.

In some embodiments, the variant differs from SEQ ID NO: 21 by 4 substitutions or 1 substitution. In some embodiments, the variant differs from SEQ ID NO: 21 by 1 substitution.

In some embodiments, at least one amino acid in position 7, 9 or 10 of SEQ ID NO: 21 is substituted with an amino acid having a positively charged side chain and/or an amino acid having an aromatic side chain. In some embodiments, exactly one amino acid in position 7, 9 or 10 of SEQ ID NO: 21 is substituted with an amino acid having a positively charged side chain and/or an amino acid having an aromatic side chain.

In some embodiments, any substitutions are at amino acid positions selected from positions 7, 9, 10, 13, 15 and 30 of SEQ ID NO: 21.

In some embodiments, the variant:
(i) differs from SEQ ID NO: 21 by 1 substitution, wherein an amino acid at position 7 or 9 of SEQ ID NO: 21 is substituted with an amino acid having a positively charged side chain and/or an amino acid having an aromatic side chain, or
(ii) differs from SEQ ID NO: 21 by 4 substitutions, wherein exactly one amino acid in position 7 or 10 of SEQ ID NO: 21 is substituted with an amino acid having a positively charged side chain and/or an amino acid having an aromatic side chain.

In some embodiments, the variant:
(i) differs from SEQ ID NO: 21 by 1 substitution, wherein an amino acid at position 7 or 9 of SEQ ID NO: 21 is substituted with an amino acid having a positively charged side chain and/or an amino acid having an aromatic side chain, or
(ii) differs from SEQ ID NO: 21 by 4 substitutions, wherein exactly one amino acid in position 7 or 10 of SEQ ID NO: 21 is substituted with an amino acid having a positively charged side chain and/or an amino acid having an aromatic side chain, and wherein the three other substitutions are at positions 13, 15 and 30 of SEQ ID NO: 21.

In some embodiments, the variant:
(i) differs from SEQ ID NO: 21 by 1 substitution, wherein an amino acid at position 7 or 9 of SEQ ID NO: 21 is substituted with an amino acid having a positively charged side chain and/or an amino acid having an aromatic side chain, or
(ii) differs from SEQ ID NO: 21 by 4 substitutions, wherein exactly one amino acid in position 7 or 10 of SEQ ID NO: 21 is substituted with an amino acid having a positively charged side chain and/or an amino acid having an aromatic side chain, wherein the three other substitutions are at positions 13, 15 and 30 of SEQ ID NO: 21, and wherein
   (a) the amino acid at position 13 is A; and/or
   (b) the amino acid at position 15 is S; and/or
   (c) the amino acid at position 30 is G. In some embodiments, the variant:
(i) differs from SEQ ID NO: 21 by 1 substitution, wherein an amino acid at position 7 or 9 of SEQ ID NO: 21 is substituted with an amino acid selected from the group consisting of H, R, Orn, 2,3-diaminopropanoyl, 2,4-diaminobutanoyl, 2-amino-3-guanidinopropionyl and Y, or
(ii) differs from SEQ ID NO: 21 by 4 substitutions, wherein exactly one amino acid in position 7 or 10 of SEQ ID NO: 21 is substituted with an amino acid selected from the group consisting of R and 2,4-diaminobutanoyl.

In some embodiments, the variant:
(i) differs from SEQ ID NO: 21 by 1 substitution, wherein an amino acid at position 7 of SEQ ID NO: 21 is substituted with an amino acid selected from the group consisting of H, R, Orn, 2,3-diaminopropanoyl, 2,4-diaminobutanoyl, 2-amino-3-guanidinopropionyl and Y or an amino acid at position 9 of SEQ ID NO: 21 is substituted with Orn, or
(ii) differs from SEQ ID NO: 21 by 4 substitutions, wherein exactly one amino acid at position 7 of SEQ ID NO: 21 is substituted with an amino acid selected from the group consisting of R and 2,4-diaminobutanoyl or exactly one amino acid at position 10 of SEQ ID NO: 21 is substituted with R.

### Sequences

In preferred embodiments, the ion channel blocker of the invention comprises or consists of one of the following sequences:

| | SEQ |
|---|---|
| | ID |
| Sequence | NO |
| P[Nle]E[Nle]RC[2,4-Diaminobutanoyl]ASVECKQKC | 10 |
| LAAIGSIFGKC[Nle]NKKCKCYPR | |
| P[Nle]E[Nle]RC[Orn]ASVECKQKCLAAIGSIFGKC[Nle] | 11 |
| NKKCKCYPR | |
| P[Nle]E[Nle]RC[2-Amino-3-guanidinopropionyl] | 12 |
| ASVECKQKCLAAIGSIFGKC[Nle]NKKCKCYPR | |
| P[Nle]E[Nle]RCHASVECKQKCLAAIGSIFGKC[Nle]NKKC | 13 |
| KCYPR | |
| P[Nle]E[Nle]RCYASVECKQKCLAAIGSIFGKC[Nle]NKKC | 14 |
| KCYPR | |
| P[Nle]E[Nle]RCSA[Orn]VECKQKCLAAIGSIFGKC[Nle] | 15 |
| NKKCKCYPR | |
| P[Nle]E[Nle]RC[2,3-Diaminopropanoyl]ASVECKQK | 16 |
| CLAAIGSIFGKC[Nle]NKKCKCYPR | |
| P[Nle]E[Nle]RCSA[2,3-Diaminopropanoyl]VECKQK | 17 |
| CLAAIGSIFGKC[Nle]NKKCKCYPR | |
| P[Nle]E[Nle]RC[2,4-Diaminobutanoyl]ASVECAQSC | 18 |
| LAAIGSIFGKC[Nle]NGKCKCYPR | |
| P[Nle]E[Nle]RCRASVECAQSCLAAIGSIFGKC[Nle]NGKC | 19 |
| KCYPR | |
| P[Nle]E[Nle]RCSASRECAQSCLAAIGSIFGKC[Nle]NGKC | 20 |
| KCYPR | |

In preferred embodiments, the ion channel blocker of the invention comprises SEQ ID NO 10 or SEQ ID NO 12. In preferred embodiments, the ion channel blocker of the invention consists of SEQ ID NO 10 or SEQ ID NO 12.

### Compounds

In preferred embodiments, the ion channel blocker of the invention comprises or consists of one of the following compounds:

| Cpd | | SEQ |
|---|---|---|
| No. | Sequence | ID NO |
| 1 | H-P[Nle]E[Nle]RC(1)[2,4-Diaminobutanoyl]ASVEC(2)KQKC | 10 |
| | (3)LAAIGSIFGKC(1)[Nle]NKKC(2)KC(3)YPR-OH | |
| 2 | H-P[Nle]E[Nle]RC(1)[Orn]ASVEC(2)KQKC(3)LAAIGSIFGKC(1) | 11 |
| | [Nle]NKKC(2)KC(3)YPR-OH | |
| 3 | H-P[Nle]E[Nle]RC(1)[2-Amino-3-guanidinopropionyl]ASVE | 12 |
| | C(2)KQKC(3)LAAIGSIFGKC(1)[Nle]NKKC(2)KC(3)YPR-OH | |
| 4 | H-P[Nle]E[Nle]RC(1)HASVEC(2)KQKC(3)LAAIGSIFGKC(1)[Nle] | 13 |
| | NKKC(2)KC(3)YPR-OH | |
| 5 | H-P[Nle]E[Nle]RC(1)YASVEC(2)KQKC(3)LAAIGSIFGKC(1)[Nle] | 14 |
| | NKKC(2)KC(3)YPR-OH | |
| 6 | H-P[Nle]E[Nle]RC(1)SA[Orn]VEC(2)KQKC(3)LAAIGSIFGKC(1) | 15 |
| | [Nle]NKKC(2)KC(3)YPR-OH | |
| 7 | H-P[Nle]E[Nle]RC(1)[2,3-Diaminopropanoyl]ASVEC(2)KQKC | 16 |
| | (3)LAAIGSIFGKC(1)[Nle]NKKC(2)KC(3)YPR-OH | |
| 8 | H-P[Nle]E[Nle]RC(1)SA[2,3-Diaminopropanoyl]VEC(2)KQKC | 17 |
| | (3)LAAIGSIFGKC(1)[Nle]NKKC(2)KC(3)YPR-OH | |
| 9 | H-P[Nle]E[Nle]RC(1)[2,4-Diaminobutanoyl]ASVEC(2)AQSC | 18 |
| | (3)LAAIGSIFGKC(1)[Nle]NGKC(2)KC(3)YPR-OH | |
| 10 | H-P[Nle]E[Nle]RC(1)[2,4-Diaminobutanoyl]ASVEC(2)AQSC | 18 |
| | (3)LAAIGSIFGKC(1)[Nle]NGKC(2)KC(3)YPR-[NH2] | |
| 11 | H-P[Nle]E[Nle]RC(1)RASVEC(2)AQSC(3)LAAIGSIFGKC(1)[Nle] | 19 |
| | NGKC(2)KC(3)YPR-[NH2] | |
| 12 | H-P[Nle]E[Nle]RC(1)SASREC(2)AQSC(3)LAAIGSIFGKC(1)[Nle] | 20 |
| | NGKC(2)KC(3)YPR-[NH2] | |

In preferred embodiments, the ion channel blocker of the invention comprises Cpd No. 1 or Cpd No. 3. In preferred embodiments, the ion channel blocker of the invention consists of Cpd No. 1 or Cpd No. 3.

### Disclaimer

The international patent application PCT/EP2020/076187 disclosed an ion channel blocker comprising or consisting of the sequence of SEQ ID NO: 2, 3, 4, 5, 6, 7, 8 or 9 (SEQ ID NO: 25, 27, 42, 47, 63, 143, 144 and 145 respectively in PCT/EP2020/076187).

International patent application PCT/EP2020/076187 also disclosed the following compounds:

| Cpd no. in | | Present | Present |
|---|---|---|---|
| PCT/EP2020/ | | SEQ ID | Cpd |
| 076187 | Compound sequence | NO | No. |
| 25 | H-NMDMRCKASVECKQKCLKAIGSIFGKCMNKKCKCYPR-NH₂ | 2 | 29 |
| 27 | H-NMDMRCSASRECKQKCLKAIGSIFGKCMNKKCKCYPR-NH₂ | 3 | 31 |
| 42 | H-N[Nle]D[Nle]RCRASVECKQKCLKAIGSIFGKC[Nle] | 4 | 46 |
| | NKKCKCYPR-NH₂ | | |
| 47 | H-N[Nle]D[Nle]RCSHSVECKQKCLKAIGSIFGKC[Nle] | 5 | 51 |
| | NKKCKCYPR-NH₂ | | |
| 63 | H-N[Nle]D[Nle]RCSASKECKQKCLKAIGSIFGKC[Nle] | 6 | 63 |
| | NKKCKCYPR-NH₂ | | |
| 143 | H-P[Nle]E[Nle]RCFASVECKQKCLAAIGSIFGKC[Nle] | 7 | None |
| 144 | H-P[Nle]E[Nle]RCSYSVECKQKCLAAIGSIFGKC[Nle] | 8 | None |
| | NKKCKCYPR-OH | | |
| 145 | H-P[Nle]E[Nle]RCSAFVECKQKCLAAIGSIFGKC[Nle] | 9 | None |
| | NKKCKCYPR-OH | | |

International patent application PCT/EP2020/076187 did not disclose any other specific compounds comprising or consisting of the sequence of SEQ ID NO: 2, 3, 4, 5, 6, 7, 8 or 9. For example, PCT/EP2020/076187 did not disclose SEQ ID NO: 2, 3, 4, 5 or 6 with a C-terminal hydroxyl (—OH) group, or SEQ ID NO: 7, 8 or 9 with a C-terminal amino group (—NH₂).

Accordingly, the ion channel blocker of the invention comprises a variant of SEQ ID NO: 1, wherein the variant does not comprise any of the following sequences:

SEQ ID NO: 2
NMDMRCKASVECKQKCLKAIGSIFGKCMNKKCKCYPR
SEQ ID NO: 3
NMDMRCSASRECKQKCLKAIGSIFGKCMNKKCKCYPR
SEQ ID NO: 4
N[Nle]D[Nle]RCRASVECKQKCLKAIGSIFGKC[Nle]NKKCKCYPR
SEQ ID NO: 5
N[Nle]D[Nle]RCSHSVECKQKCLKAIGSIFGKC[Nle]NKKCKCYPR
SEQ ID NO: 6
N[Nle]D[Nle]RCSASKECKQKCLKAIGSIFGKC[Nle]NKKCKCYPR
SEQ ID NO: 7
P[Nle]E[Nle]RCFASVECKQKCLAAIGSIFGKC[Nle]NKKCKCYPR
SEQ ID NO: 8
P[Nle]E[Nle]RCSYSVECKQKCLAAIGSIFGKC[Nle]NKKCKCYPR
SEQ ID NO: 9
P[Nle]E[Nle]RCSAFVECKQKCLAAIGSIFGKC[Nle]NKKCKCYPR

The ion channel blocker of the invention is not any of compounds 25, 27, 42, 47, 63, 143, 144 or 145 disclosed in the international patent application PCT/EP2020/076187.

In some embodiments of the ion channel blocker of the invention, the variant of SEQ ID NO: 1 does not comprise or consist of the sequence of SEQ ID NO: 2, 3, 4, 5, 6, 7, 8 or 9. In some embodiments, the ion channel blocker of the invention does not comprise any of the sequences disclosed in international patent application PCT/EP2020/076187. In some embodiments, the ion channel blocker of the invention is not any of the compounds disclosed in international patent application PCT/EP2020/076187.

### Methods of Synthesising Ion Channel Blocker

The ion channel blockers described herein may be synthesised by means of solid-phase or liquid-phase peptide synthesis methodology. In this context, reference may be made to WO 98/11125 and, among many others, Fields, G. B. et al., 2002, “Principles and practice of solid-phase peptide synthesis”. In: Synthetic Peptides (2nd Edition), and the Examples herein.

Alternatively, the ion channel blockers described herein may be synthesised by recombinant techniques, or by a combination of recombinant techniques and peptide chemistry.

Thus, in one aspect the invention provides a method of synthesising an ion channel blocker of the invention, the method comprising:
(a) synthesising the peptide by means of solid-phase or liquid-phase peptide synthesis methodology and recovering the peptide thus obtained;
(b) expressing the peptide from a nucleic acid construct that encodes the peptide and recovering the expression product; or
(c) expressing a precursor peptide from a nucleic acid construct that encodes the precursor peptide sequence, recovering the expression product, and modifying the precursor peptide to yield an ion channel blocker of the invention.

The precursor peptide may be modified by introduction of one or more non-proteinogenic amino acids (e.g. Nle), introduction of the appropriate terminal groups R¹ and R², etc.

Expression of the peptide or precursor peptide from a nucleic acid encoding the peptide or precursor peptide may be performed in a cell or a cell-free expression system comprising such a nucleic acid. Such expression typically requires that the peptide or precursor peptide is composed entirely of proteinogenic amino acids (i.e. the 20 amino acids encoded by the standard genetic code.)

For recombinant expression, the nucleic acid fragments encoding the precursor peptide will normally be inserted in suitable vectors to form cloning or expression vectors. The vectors can, depending on purpose and type of application, be in the form of plasmids, phages, cosmids, mini-chromosomes, or virus, but also naked DNA which is only expressed transiently in certain cells is an important vector. Preferred cloning and expression vectors (plasmid vectors) are capable of autonomous replication, thereby enabling high copy-numbers for the purposes of high-level expression or high-level replication for subsequent cloning.

In general outline, an expression vector comprises the following features in the 5′→3′ direction and in operable linkage: a promoter for driving expression of the nucleic acid fragment, optionally a nucleic acid sequence encoding a leader peptide enabling secretion (to the extracellular phase or, where applicable, into the periplasm), the nucleic acid fragment encoding the precursor peptide, and optionally a nucleic acid sequence encoding a terminator. They may comprise additional features such as selectable markers and origins of replication. When operating with expression vectors in producer strains or cell lines it may be preferred that the vector is capable of integrating into the host cell genome. The skilled person is very familiar with suitable vectors and is able to design one according to their specific requirements.

The vectors of the invention are used to transform host cells to produce the peptide or precursor peptide. Such transformed cells can be cultured cells or cell lines used for propagation of the nucleic acid fragments and vectors, and/or used for recombinant production of the precursor peptides.

Preferred transformed cells are micro-organisms such as bacteria [such as the species Escherichia (e.g. E. coli), Bacillus (e.g. Bacillus subtilis), Salmonella, or Mycobacterium (preferably non-pathogenic, e.g. M. bovis BCG), yeasts (e.g., Saccharomyces cerevisiae and Pichia pastoris), and protozoans. Alternatively, the transformed cells may be derived from a multicellular organism, i.e. it may be fungal cell, an insect cell, an algal cell, a plant cell, or an animal cell such as a mammalian cell. For the purposes of cloning and/or optimised expression it is preferred that the transformed cell is capable of replicating the nucleic acid fragment of the invention. Cells expressing the nucleic fragment can be used for small-scale or large-scale preparation of the peptides of the invention.

When producing the peptide or precursor peptide by means of transformed cells, it is convenient, although far from essential, that the expression product is secreted into the culture medium.

### Pharmaceutical Composition

In another aspect the invention provides a pharmaceutical composition comprising an ion channel blocker, or pharmaceutically acceptable salt thereof, of the invention. In some embodiments, the pharmaceutical composition of the invention comprises an ion channel blocker, or pharmaceutically acceptable salt thereof, of the invention and a pharmaceutically acceptable carrier, excipient or vehicle.

Any of the ion channel blockers of the invention may be in the form of a pharmaceutically acceptable salt. All references to “an ion channel blocker of the invention” herein should be considered to encompass any pharmaceutically acceptable salt thereof, regardless of whether “pharmaceutically acceptable salt” is explicitly recited. The ion channel blocker may also be referred to as a “solvate” meaning a complex of defined stoichiometry formed between a solute (a peptide or pharmaceutically acceptable salt thereof according to the invention) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

Accordingly, the compounds of the present invention, or salts thereof, especially pharmaceutically acceptable salts thereof, may be formulated as compositions or pharmaceutical compositions prepared for storage or administration, and which comprise a therapeutically effective amount of a compound of the invention, or a salt thereof.

Suitable salts formed with bases include metal salts, such as alkali metal or alkaline earth metal salts, for example sodium, potassium or magnesium salts; ammonia salts and organic amine salts, such as those formed with morpholine, thiomorpholine, piperidine, pyrrolidine, a lower mono-, di- or tri-alkylamine (e.g., ethyl-tert-butyl-, diethyl-, diisopropyl-, triethyl-, tributyl- or dimethylpropylamine), or a lower mono-, di- or tri-(hydroxyalkyl)amine (e.g., mono-, di- or triethanolamine). Internal salts may also be formed. Similarly, when a compound of the present invention contains a basic moiety, salts can be formed using organic or inorganic acids. For example, salts can be formed from the following acids: formic, acetic, propionic, butyric, valeric, caproic, oxalic, lactic, citric, tartaric, succinic, fumaric, maleic, malonic, mandelic, malic, phthalic, hydrochloric, hydrobromic, phosphoric, nitric, sulphuric, benzoic, carbonic, uric, methanesulphonic, naphthalenesulphonic, benzenesulphonic, toluenesulphonic, p-toluenesulphonic (i.e. 4-methylbenzene-sulphonic), camphorsulphonic, 2-aminoethanesulphonic, aminomethylphosphonic and trifluoromethanesulphonic acid (the latter also being denoted triflic acid), as well as other known pharmaceutically acceptable acids. Amino acid addition salts can also be formed with amino acids, such as lysine, glycine, or phenylalanine.

In some embodiments, a pharmaceutical composition of the invention is one wherein the compound is in the form of a pharmaceutically acceptable acid addition salt.

As will be apparent to one skilled in the medical art, a “therapeutically effective amount” of a compound or pharmaceutical composition of the present invention will vary depending upon, inter alia, the age, weight and/or gender of the subject (patient) to be treated. Other factors that may be of relevance include the physical characteristics of the specific patient under consideration, the patient's diet, the nature of any concurrent medication, the particular compound(s) employed, the particular mode of administration, the desired pharmacological effect(s) and the particular therapeutic indication. Because these factors and their relationship in determining this amount are well known in the medical arts, the determination of therapeutically effective dosage levels to achieve the desired therapeutic effect will be within the ambit of the skilled person.

As used herein, the term “a therapeutically effective amount” refers to an amount which reduces symptoms of a given condition or pathology, and preferably which normalizes physiological responses in an individual with that condition or pathology. Reduction of symptoms or normalization of physiological responses can be determined using methods routine in the art and may vary with a given condition or pathology. In one aspect, a therapeutically effective amount of a compound of the invention, or a pharmaceutical composition, is an amount which restores a measurable physiological parameter to substantially the same value (preferably to within 30%, more preferably to within 20%, and still more preferably to within 10% of the value) of the parameter in an individual without the condition or pathology in question.

In one embodiment of the invention, administration of a compound or pharmaceutical composition of the present invention is commenced at lower dosage levels, with dosage levels being increased until the desired effect of preventing/treating the relevant medical indication is achieved. This would define a therapeutically effective amount. For the compounds of the present invention, alone or as part of a pharmaceutical composition, such human doses of the active compound may be between about 0.01 pmol/kg and 500 μmol/kg body weight, between about 0.01 pmol/kg and 300 μmol/kg body weight, between 0.01 pmol/kg and 100 μmol/kg body weight, between 0.1 pmol/kg and 50 μmol/kg body weight, between 1 pmol/kg and 10 μmol/kg body weight, between 5 pmol/kg and 5 μmol/kg body weight, between 10 pmol/kg and 1 μmol/kg body weight, between 50 pmol/kg and 0.1 μmol/kg body weight, between 100 pmol/kg and 0.01 μmol/kg body weight, between 0.001 μmol/kg and 0.5 μmol/kg body weight, between 0.05 μmol/kg and 0.1 μmol/kg body weight.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

### Medical Use and Methods of Treatment

In a further aspect, the invention provides an ion channel blocker or pharmaceutically acceptable salt of the invention for use in a method of medical treatment. In other words, the invention provides a method of medical treatment comprising administering the ion channel blocker or pharmaceutically acceptable salt of the invention to a subject.

Medical uses of the ion channel blocker or pharmaceutically acceptable salt of the invention are described herein. In all cases, the medical use described may also be phrased as a method of treatment comprising administering the ion channel blocker or pharmaceutically acceptable salt of the invention to a subject in need of treatment.

The terms “patient”, “subject” and “individual” may be used interchangeably herein and refer to either a human or a non-human animal. These terms include mammals such as humans, primates, livestock animals (e.g., bovines and porcines), companion animals (e.g., canines and felines) and rodents (e.g., mice and rats).

As discussed above, blockers of Kv1.3 have been shown to inhibit proliferation of activated T cells and to have a beneficial effect in various experimental models of disease. Without wishing to be bound by theory, it is believed that cellular efflux of potassium via the Kv1.3 channel is required to sustain calcium influx required for T-cell activation.

Kv1.3 is overexpressed in Gad5/insulin-specific T cells from patients with new onset type 1 diabetes, in myelin-specific T cells from MS patients and in T cells from the synovium of rheumatoid arthritis patients (Beeton et al., Proc Natl Acad Sci USA 103:17414-9, 2006), in breast cancer specimens (Abdul et al., Anticancer Res 23:3347, 2003) and prostate cancer cell lines (Fraser et al., Pflugers Arch 446:559, 2003).

Positive outcomes in animal models with Kv1.3 blockers have been described in hypersensitivity models to ovalbumin and tetanus toxoid (Beeton et al., Mol Pharmacol 67:1369, 2005; Koo et al., Clin Immunol 197:99, 1999), models for multiple sclerosis such as rat adoptive-transfer experimental autoimmune encephalomyelitis (AT-EAE) model (Beeton et al., Proc Natl Acad Sci USA 103:17414-9, 2006), inflammatory bone resorption model (Valverde et al., J Bone Mineral Res 19:155, 2004), models for arthritis (Beeton et al., Proc Natl Acad Sci 103: 17414, 2006; Tarcha et al., J. Pharmacol. Exp. Ther. 342: 642, 2012) and obesity, diabetes and metabolic disorders (Xu et al., Hum Mol Genet 12:551, 2003; Xu et al., Proc Natl Acad Sci 101: 3112, 2004).

Topical application of Kv1.3 blockers has been proposed for the treatment of skin and mucosal inflammation.

### Treatment of Inflammation

In some embodiments the invention provides an ion channel blocker or pharmaceutically acceptable salt of the invention for use in a method of inhibiting or reducing inflammation.

In some embodiments the invention provides an ion channel blocker or pharmaceutically acceptable salt for use in the treatment of an inflammatory condition or disorder.

An inflammatory condition or disorder may be any condition or disorder in which reduction of inflammation is desirable, e.g. where inflammation contributes to symptoms or pathogenesis.

In some embodiments the inflammatory condition or disorder is an autoimmune disorder, allergy or hypersensitivity, allograft rejection, or graft versus host disease.

In some embodiments the inflammatory condition or disease is hay fever, asthma, anaphylaxis, allergic rhinitis, urticaria, eczema, alopecia areata, dermatomyositis, inclusion body myositis, polymyositis, ankylosing spondylitis, vasculitis, arthritis (including rheumatoid arthritis, osteoarthritis, psoriatic arthritis), Sjogren's syndrome, systemic lupus erythematosus (SLE), uveitis, inflammatory fibrosis (e.g. scleroderma, lung fibrosis, cirrhosis), chronic obstructive pulmonary disease (COPD), hepatitis, chronic inflammatory demyelinating polyneuropathy, inflammatory bowel disease, colitis (e.g. Crohn's disease and ulcerative colitis), erythema, thyroiditis, psoriasis, atopic dermatitis, allergic contact dermatitis, scleroderma, glomerulonephritis, inflammatory bone resorption, multiple sclerosis, type 1 diabetes, transplant rejection or graft-versus-host disease.

### Metabolic Effects

Blockers of Kv1.3 may also have beneficial metabolic effects, e.g. in relation to energy homeostasis, body weight regulation, and glucose control.

The ion channel blockers described here may therefore be used for inhibiting weight gain, promoting weight loss, reducing excess body weight or treating obesity (e.g. by control of appetite, feeding, food intake, calorie intake, and/or energy expenditure), as well as in the treatment of associated disorders and health conditions including obesity linked inflammation, obesity linked gallbladder disease and obesity induced sleep apnoea.

An effect on body weight may be therapeutic or cosmetic.

The ion channel blockers may also be used for the treatment of conditions caused by or associated with impaired glucose control, including metabolic syndrome, insulin resistance, glucose intolerance, pre-diabetes, increased fasting glucose and type 2 diabetes. Some of these conditions can be associated with obesity. Their effects on these conditions may be mediated in whole or in part via an effect on body weight, or may be independent thereof.

### Treatment of Proliferating Cells and Cancer

Kv1.3 is also expressed in proliferating human and mouse smooth muscle cells. Blockers of Kv1.3 may be effective in smooth muscle proliferative disorders such as restenosis, e.g. in patients following vascular surgery (e.g. angioplasty).

In some embodiments the invention provides an ion channel blocker or pharmaceutically acceptable salt according to the invention for use in treatment of a smooth muscle proliferative disorder. In some embodiments the disorder is restenosis.

Further evidence suggests that Kv1.3 channels are involved in the activation and/or proliferation of many types of cells, including tumor cells (Bielanska et al., Curr. Cancer Drug Targets 9:904-14, 2009), microglia (Khanna et al., Am. J. Physiol. Cell Physiol. 280: C796-806, 2001) and differentiation of neuronal progenitor cells (Wang et al., J. Neurosci. 30:5020-7, 2010). Kv1.3 blockers may therefore be beneficial in the treatment of neuroinflammatory and neurodegenerative disorders such as Alzheimer's disease, multiple sclerosis (MS), Parkinson's disease and amyotrophic lateral sclerosis (ALS) (e.g. following viral infections).

In some embodiments the invention provides an ion channel blocker or pharmaceutically acceptable salt of the invention for use in treatment of cancer. In some embodiments the cancer is breast cancer, prostate cancer or lymphoma. In some embodiments the lymphoma is non-Hodgkin lymphoma (NHL). Non-Hodgkin lymphomas include T-cell NHL and B-cell NHL. Forms of B-cell NHL include diffuse large B-cell lymphoma, follicular lymphoma, Burkitt lymphoma, immunoblastic large cell lymphoma, precursor B-lymphoblastic lymphoma and mantle cell lymphoma. Forms of T-cell NHL include mycosis fungoides, anaplastic large cell lymphoma, peripheral T-cell lymphoma, precursor T-lymphoblastic lymphoma and Sézary syndrome.

### PATENT TERMINOLOGY

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well known and commonly used in the art.

All patents, published patent applications and non-patent publications referred to in this application are specifically incorporated by reference herein. In case of conflict, the present specification, including its specific definitions, will control.

Each embodiment of the invention described herein may be taken alone or in combination with one or more other embodiments of the invention.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

As used herein, the singular forms “a”, “an”, and “the” include plural referents unless the context clearly dictates otherwise.

Throughout this specification, the word “comprise”, and grammatical variants thereof, such as “comprises” or “comprising”, will be understood to imply the inclusion of a stated integer or component, or group of integers or components, but not the exclusion of any other integer or component, or group of integers or components. The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”. The term “including” is used to mean “including but not limited to”. “Including” and “including but not limited to” may be used interchangeably.

The publications discussed herein are provided solely for their disclosure prior to the filing date of the present application. Nothing herein is to be construed as an admission that such publications constitute prior art to the aspects appended hereto.

The invention will now be further described by way of Examples, which are meant to serve to assist one of ordinary skill in the art in carrying out the invention and are not intended in any way to limit the scope of the invention.

### EXAMPLES

### Example 1: General Peptide Synthesis

A list of abbreviations and suppliers is provided in the table below.

| | Abbreviation | Name | Brand/Supplier |
|---|---|---|---|
| Resins | None | TentaGel ™ PHB AA(Proct)-Fmoc | Rapp Polymere |
| | None | TentaGel ™ SRAM | Rapp Polymere |
| Amino | None | Pseudoprolines (e.g. QT, AT, FS) | Jupiter Bioscience Ltd. |
| acids | None | Fmoc-L-AA-OH | Senn Chemicals AG |
| Coupling | COMU | (1-Cyano-2-ethoxy-2- | Watson International Ltd. |
| reagents | | oxoethylidenaminooxy)dimethylamino- | |
| | | morpholino-carbenium | |
| | | hexafluorophosphate | |
| | DIC | Diisopropylcarbodiimide | Fluka/Sigma Aldrich Co. |
| | HATU | N-[(dimethylamino)-1H-1,2,3- | ChemPep Inc. |
| | | triazol[4,5-b]pyridine-1- | |
| | | ylmethylene]-N-methylmethanaminium | |
| | | hexafluorophosphate N-oxide | |
| | HOBt | Hydroxybenzotriazole | Sigma-Aldrich Co. |
| Solvents | Boc₂O | Di-tert-butyl pyrocarbonate | Advanced ChemTech |
| reagents | DCM | Dichloromethane | Prolabo (VWR) |
| | DIPEA | Diisopropylethylamine | Fluka/Sigma Aldrich Co. |
| | DMF | N,N-dimethylformamide | Taminco |
| | DODT | 3,6-dioxa-1,8-octanedithiol | Sigma-Aldrich Co. |
| | Et₂O | Diethyl ether | Prolabo (VWR) |
| | EtOH | Ethanol | CCS Healthcare AB |
| | None | Formic acid (HPLC) | Sigma-Aldrich Co. |
| | H₂O | Water, Milli-Q water | Millipore |
| | MeCN | Acetonitrile (HPLC) | Sigma-Aldrich Co. |
| | MeOH | Methanol | Sigma-Aldrich Co. |
| | NMP | N-methylpyrrolidone | Sigma-Aldrich Co. |
| | None | Piperidine | Jubliant Life Sciences Ltd. |
| | TFA | Trifluoroacetic acid (HPLC) | Chemicals Raw Materials Ltd. |
| | TIS | Triisopropylsilane | Sigma-Aldrich Co. |

### Apparatus and Synthetic Strategy

Peptides were synthesized batchwise on a peptide synthesiser, such as a CEM Liberty Peptide Synthesizer or a Symphony X Synthesizer, according to solid phase peptide synthetic procedures using 9-fluorenylmethyloxycarbonyl (Fmoc) as N-α-amino protecting group and suitable common protection groups for side-chain functionalities.

As polymeric support based resins, such as e.g. TentaGel™, was used. The synthesizer was loaded with resin that prior to usage was swelled in DMF.

### Coupling

### CEM Liberty Peptide Synthesizer

A solution of Fmoc-protected amino acid (4 equiv.) was added to the resin together with a coupling reagent solution (4 equiv.) and a solution of base (8 equiv.). The mixture was either heated by the microwave unit to 70-75° C. and coupled for 5 minutes or coupled with no heat for 60 minutes. During the coupling nitrogen was bubbled through the mixture.

### Symphony X Synthesizer

The coupling solutions were transferred to the reaction vessels in the following order: amino acid (4 equiv.), HATU (4 equiv.) and DIPEA (8 equiv.). The coupling time was 10 min at room temperature (RT) unless otherwise stated. The resin was washed with DMF (5×0.5 min). In case of repeated couplings the coupling time was in all cases 45 min at RT.

### Deprotection

### CEM Liberty Peptide Synthesizer

The Fmoc group was deprotected using piperidine in DMF or other suitable solvents. The deprotection solution was added to the reaction vessel and the mixture was heated for 30 sec. reaching approx. 40° C. The reaction vessel was drained and fresh deprotection solution was added and subsequently heated to 70-75° C. for 3 min. After draining the reaction vessel the resin was washed with DMF or other suitable solvents.

### Symphony X Synthesizer

Fmoc deprotection was performed for 2.5 minutes using 40% piperidine in DMF and repeated using the same conditions. The resin was washed with DMF (5×0.5 min).

### Cleavage

The dried peptide resin was treated with TFA and suitable scavengers for approximately 2 hours. The volume of the filtrate was reduced and the crude peptide was precipitated after addition of diethylether. The crude peptide precipitate was washed several times with diethylether and finally dried.

### HPLC Purification of the Crude Peptide

The crude peptide was purified by preparative reverse phase HPLC using a conventional HPLC apparatus, such as a Gilson GX-281 with 331/332 pump combination', for binary gradient application equipped with a column, such as 5×25 cm Gemini NX 5u C18 110A column, and a fraction collector using a flow 20-40 ml/min with a suitable gradient of buffer A (0.1% Formic acid, aq.) or A (0.1% TFA, aq.) and buffer B (0.1% Formic acid, 90% MeCN, aq.) or B (0.1% TFA, 90% MeCN, aq.). Fractions were analyzed by analytical HPLC and MS and selected fractions were pooled and lyophilized. The final product was characterized by HPLC and MS.

### Disulphide Formation

The crude or partially purified linear peptide with six cysteines was dissolved in a buffer such as sodium hydrogen carbonate (NaHCO₃) or ammonium acetate (NH₄Ac) to give a final concentration of approximate 0.1 mg/ml or 25 μM. The pH of the buffer was adjusted to pH 8.0 and the solution was stirred at room temperature under magnetic stirring and open access to the atmosphere. The progress of the reaction was determined by HPLC and was usually evaluated to be complete overnight. The solution was quenched by reducing the pH of the solution by an organic acid such as acetic acid or trifluoroacetic acid (pH<4). The solution was filtered and loaded directly on a prep-HPLC column for purification.

### Analytical HPLC

Final purities were determined by analytic HPLC (Agilent 1100/1200 series) equipped with auto sampler, degasser, 20 μl flow cell and Chromeleon software. The HPLC was operated with a flow of 1.2 ml/min at 40° C. using an analytical column, such as Kinetex 2.6 μm XB-C18 100A 100×4.6 mm column. The compound was detected and quantified at 215 nm. Buffers A (0.1% TFA, aq.) and buffer B (0.1% TFA, 90% MeCN, aq.).

### Mass Spectroscopy

Final MS analysis was performed on a conventional mass spectrometer, e.g. Waters Xevo G2 Tof, equipped with electrospray detector with lock-mass calibration and MassLynx software. It was operated in positive mode using direct injection and a cone voltage of 15V (1 TOF), 30 V (2 TOF) or 45 V (3 TOF) as specified on the chromatogram. Precision was 5 ppm with a typical resolution of 15,000-20,000.

### Compounds

Compounds synthesized are shown in Table 1.

TABLE 1

| Cpd | | SEQ |
|---|---|---|
| No. | Sequence | ID NO |
| 1 | H-P[Nle]E[Nle]RC(1)[2,4-Diaminobutanoyl]ASVEC(2)KQKC | 10 |
| | (3)LAAIGSIFGKC(1)[Nle]NKKC(2)KC(3)YPR-OH | |
| 2 | H-P[Nle]E[Nle]RC(1)[Orn]ASVEC(2)KQKC(3)LAAIGSIFGKC(1) | 11 |
| | [Nle]NKKC(2)KC(3)YPR-OH | |
| 3 | H-P[Nle]E[Nle]RC(1)[2-Amino-3-guanidinopropionyl]ASVE | 12 |
| | C(2)KQKC(3)LAAIGSIFGKC(1)[Nle]NKKC(2)KC(3)YPR-OH | |
| 4 | H-P[Nle]E[Nle]RC(1)HASVEC(2)KQKC(3)LAAIGSIFGKC(1)[Nle] | 13 |
| | NKKC(2)KC(3)YPR-OH | |
| 5 | H-P[Nle]E[Nle]RC(1)YASVEC(2)KQKC(3)LAAIGSIFGKC(1)[Nle] | 14 |
| | NKKC(2)KC(3)YPR-OH | |
| 6 | H-P[Nle]E[Nle]RC(1)SA[Orn]VEC(2)KQKC(3)LAAIGSIFGKC(1) | 15 |
| | [Nle]NKKC(2)KC(3)YPR-OH | |
| 7 | H-P[Nle]E[Nle]RC(1)[2,3-Diaminopropanoyl]ASVEC(2)KQKC | 16 |
| | (3)LAAIGSIFGKC(1)[Nle]NKKC(2)KC(3)YPR-OH | |
| 8 | H-P[Nle]E[Nle]RC(1)SA[2,3-Diaminopropanoyl]VEC(2)KQKC | 17 |
| | (3)LAAIGSIFGKC(1)[Nle]NKKC(2)KC(3)YPR-OH | |
| 9 | H-P[Nle]E[Nle]RC(1)[2,4-Diaminobutanoyl]ASVEC(2)AQSC | 18 |
| | (3)LAAIGSIFGKC(1)[Nle]NGKC(2)KC(3)YPR-OH | |
| 10 | H-P[Nle]E[Nle]RC(1)[2,4-Diaminobutanoyl]ASVEC(2)AQSC | 18 |
| | (3)LAAIGSIFGKC(1)[Nle]NGKC(2)KC(3)YPR-[NH2] | |
| 11 | H-P[Nle]E[Nle]RC(1)RASVEC(2)AQSC(3)LAAIGSIFGKC(1)[Nle] | 19 |
| | NGKC(2)KC(3)YPR-[NH2] | |
| 12 | H-P[Nle]E[Nle]RC(1)SASREC(2)AQSC(3)LAAIGSIFGKC(1)[Nle] | 20 |
| | NGKC(2)KC(3)YPR-[NH2] | |

The following compounds were also synthesised for use as reference compounds:

TABLE 2

| | Cpd No. in | | SEQ |
|---|---|---|---|
| Cpd | PCT/EP2020/ | | ID |
| No. | 076187 | Sequence | NO |
| 13 | 98 | H-P[Nle]E[Nle]RC(1)SASVEC(2)KQKC(3)LAAIGSIFG | 21 |
| | | KC(1)[Nle]NKKC(2)KC(3)YPR-[NH₂] | |
| 14 | 97 | H-P[Nle]E[Nle]RC(1)SASVEC(2)KQKC(3)LAAIGSIFG | 21 |
| | | KC(1)[Nle]NKKC(2)KC(3)YPR-OH | |
| 15 | 1 | H-QMDMRCSASVECKQKCLKAIGSIFGKCMNKKCKCYPR-OH | 1 |
| 16 | 2 | H-QMDMRCSASVECKQKCLKAIGRGFGKCMNKKCKCYPR-OH | 22 |
| 17 | 3 | H-NMDMRCSASVECKQKCLKAIGSIFGKCMNKKCKCYPR-OH | 23 |
| 18 | 5 | H-MDMRCSASVECKQKCLKAIGSIFGKCMNKKCKCYPR-OH | 24 |
| 19 | 8 | H-NIDMRCSASVECKQKCLKAIGSIFGKCMNKKCKCYPR-OH | 25 |
| 20 | 12 | H-NMEMRCSASVECKQKCLKAIGSIFGKCMNKKCKCYPR-OH | 26 |
| 21 | 16 | H-NMDMRCSASVECKQKCLKAIGSIFGKC[Nle]NKKCKCYPR- | 27 |
| | | NH₂ | |
| 22 | 17 | H-NMDMRCSASVECKVKCLKAIGSIFGKCMNKKCKCYPR-NH₂ | 28 |
| 23 | 18 | H-NMDMRCSASVECKQLCLKAIGSIFGKCMNKKCKCYPR-NH₂ | 29 |
| 24 | 19 | H-NMDMRCSASVECKQKCKKAIGSIFGKCMNKKCKCYPR-NH₂ | 30 |
| 25 | 20 | H-NMDMRCSASVECKQKCLDAIGSIFGKCMNKKCKCYPR-NH₂ | 31 |
| 26 | 21 | H-NMDMRCSASVECKQKCLKAIRSIFGKCMNKKCKCYPR-NH₂ | 32 |
| 27 | 22 | H-NMDMRCSASVECKQKCLKAIESIFGKCMNKKCKCYPR-NH₂ | 33 |
| 28 | 23 | H-NMDMRCSASVECKQKCLKAIGSIFGKCMNKKCKCYPRRRTA- | 34 |
| | | NH₂ | |
| 29 | 25 | H-NMDMRCKASVECKQKCLKAIGSIFGKCMNKKCKCYPR-NH₂ | 2 |
| 30 | 26 | H-NMDMRCSISVECKQKCLKAIGSIFGKCMNKKCKCYPR-NH₂ | 35 |
| 31 | 27 | H-NMDMRCSASRECKQKCLKAIGSIFGKCMNKKCKCYPR-NH₂ | 3 |
| 32 | 28 | H-NMDMRCSASVQCKQKCLKAIGSIFGKCMNKKCKCYPR-NH₂ | 36 |
| 33 | 29 | H-NMDMRCSASVECLQKCLKAIGSIFGKCMNKKCKCYPR-NH₂ | 37 |
| 34 | 30 | H-NMDMRCSASVECAQKCLKAIGSIFGKCMNKKCKCYPR-NH₂ | 38 |
| 35 | 31 | H-NMDMRCSASVECKEKCLKAIGSIFGKCMNKKCKCYPR-NH₂ | 39 |
| 36 | 32 | H-NMDMRCSASVECKLKCLKAIGSIFGKCMNKKCKCYPR-NH₂ | 40 |
| 37 | 33 | H-NMDMRCSASVECKQKCLKAIHSIFGKCMNKKCKCYPR-NH₂ | 41 |
| 38 | 34 | H-NMDMRCSASVECKQKCLKAIGSKFGKCMNKKCKCYPR-NH₂ | 42 |
| 39 | 35 | H-NMDMRCSASVECKQKCLKAIGSRFGKCMNKKCKCYPR-NH₂ | 43 |
| 40 | 36 | H-NMDMRCSASVECKQKCLKAIGSIFGKCMNGKCKCYPR-NH₂ | 44 |
| 41 | 37 | H-NMDMRCSASVECKQKCLKAIGSIFGKCMNKKCHCYPR-NH₂ | 45 |
| 42 | 38 | H-NMDMRCSASVECKQKCLKAIGSIFGKCMNKKCVCYPR-NH₂ | 46 |
| 43 | 39 | H-N[Nle]D[Nle]RCSASVECKQKCLKAIGSIFGKCMNKKCK | 47 |
| | | CYPR-NH₂ | |
| 44 | 40 | H-N[Nle]D[Nle]RCSASVECKQKCLKAIGSIFGKC[Nle]N | 48 |
| | | KKCKCYPR-NH₂ | |
| 45 | 41 | H-P[Nle]D[Nle]RCSASVECKQKCLKAIGSIFGKC[Nle]N | 49 |
| | | KKCKCYPR-NH₂ | |
| 46 | 42 | H-N[Nle]D[Nle]RCRASVECKQKCLKAIGSIFGKC[Nle]N | 4 |
| | | KKCKCYPR-NH₂ | |
| 47 | 43 | H-N[Nle]D[Nle]RCSASVECEQKCLKAIGSIFGKC[Nle]N | 50 |
| | | KKCKCYPR-NH₂ | |
| 48 | 44 | H-N[Nle]D[Nle]RCSASVECQQKCLKAIGSIFGKC[Nle]N | 51 |
| | | KKCKCYPR-NH₂ | |
| 49 | 45 | H-N[Nle]D[Nle]RCSASVECKKKCLKAIGSIFGKC[Nle]N | 52 |
| | | KKCKCYPR-NH₂ | |
| 50 | 46 | H-N[Nle]S[Nle]RCSASVECKQKCLKAIGSIFGKC[Nle]N | 53 |
| | | KKCKCYPR-NH₂ | |
| 51 | 47 | H-N[Nle]D[Nle]RCSHSVECKQKCLKAIGSIFGKC[Nle]N | 5 |
| | | KKCKCYPR-NH₂ | |
| 52 | 48 | H-N[Nle]D[Nle]RCSASVECKQSCLKAIGSIFGKC[Nle]N | 54 |
| | | KKCKCYPR-NH₂ | |
| 53 | 49 | H-N[Nle]D[Nle]RCSASVECKQKCKAIGSIFGKC[Nle]NK | 55 |
| | | KCKCYPR-NH₂ | |
| 54 | 50 | H-NMDMRCSASVECKQKCYKAIGSIFGKCMNKKCKCYPR-NH₂ | 56 |
| 55 | 51 | H-NMDMRCSASVECKQKCRKAIGSIFGKCMNKKCKCYPR-NH₂ | 57 |
| 56 | 52 | H-NMDMRCSASVECKQKCLAAIGSIFGKCMNKKCKCYPR-NH₂ | 58 |
| 57 | 53 | H-NMDMRCSASVECKQKCLYAIGSIFGKCMNKKCKCYPR-NH₂ | 59 |
| 58 | 54 | H-NMDMRCSASVECKQKCLAIGSIFGKCMNKKCKCYPR-NH₂ | 60 |
| 59 | 55 | H-NMDMRCSASVECKQKCLKYIGSIFGKCMNKKCKCYPR-NH₂ | 61 |
| 60 | 56 | H-NMDMRCSASVECKQKCLKRIGSIFGKCMNKKCKCYPR-NH₂ | 62 |
| 61 | 58 | H-NMDMRCSASVECKQKCLKAYGSIFGKCMNKKCKCYPR-NH₂ | 63 |
| 62 | 62 | H-N[Nle]D[Nle]RCSASVECKQKCLKAIGSPFGKC[Nle]N | 64 |
| | | KKCKCYPR-NH₂ | |
| 63 | 63 | H-N[Nle]D[Nle]RCSASKECKQKCLKAIGSIFGKC[Nle]N | 6 |
| | | KKCKCYPR-NH₂ | |
| 64 | 64 | H-H[Nle]D[Nle]RCSASVECKQKCLKAIGSIFGKC[Nle]N | 65 |
| | | KKCKCYPR-NH₂ | |
| 65 | 65 | H-Y[Nle]D[Nle]RCSASVECKQKCLKAIGSIFGKC[Nle]N | 66 |
| | | KKCKCYPR-NH₂ | |
| 66 | 66 | H-S[Nle]D[Nle]RCSASVECKQKCLKAIGSIFGKC[Nle]N | 67 |
| | | KKCKCYPR-NH₂ | |
| 67 | 67 | H-V[Nle]D[Nle]RCSASVECKQKCLKAIGSIFGKC[Nle]N | 68 |
| | | KKCKCYPR-NH₂ | |
| 68 | 68 | H-S[Nle]D[Nle]RCSA[Abu]VECKQKCLKAIGSIFGKCMN | 69 |
| | | KKCKCYPR-NH₂ | |
| 69 | 69 | H-S[Nle]D[Nle]RCSASVECKQKCLKAIGSIFG[homo- | 70 |
| | | Lys]CMNKKCKCYPR-NH₂ | |
| 70 | 70 | H-S[Nle]D[Nle]RCSASVECKQKCLKAIGSIFGKCMNKKCK | 71 |
| | | C[F(4-NH₂)]PR-NH₂ | |
| 71 | 71 | H-S[Nle]D[Nle]RCSASVECGQKCLKAIGSIFGKCMNKKCK | 72 |
| | | CYPR-NH₂ | |
| 72 | 72 | H-S[Nle]D[Nle]RCSASVECVQKCLKAIGSIFGKCMNKKCK | 73 |
| | | CYPR-NH₂ | |
| 73 | 73 | H-S[Nle]D[Nle]RCSASVECK[2-Amino-5-carboxy- | 74 |
| | | pentanoyl]KCLKAIGSIFGKCMNKKCKCYPR-NH₂ | |
| 74 | 74 | H-S[Nle]D[Nle]RCSASVECKQKCLKAIGSIFGKCMNKKCK | 75 |
| | | CYQ-NH₂ | |
| 75 | 75 | H-S[Nle]D[Nle]RCSASVECKQKCLKAIGSIFGKCMNKKCRC | 76 |
| | | YPR-NH₂ | |
| 76 | 76 | H-S[Nle]DERCSASVECKQKCLKAIGSIFGKCMNKKCKCYPR- | 77 |
| | | NH₂ | |
| 77 | 78 | H-S[Nle]D[Nle]RCSASVECKQKCLVAIGSIFGKCMNKKCKC | 78 |
| | | YPR-NH₂ | |
| 78 | 79 | H-S[Nle]D[Nle]RCSASVECAQSCLKAIGSIFGKCMNKKCKC | 79 |
| | | YPR-NH₂ | |
| 79 | 80 | H-S[Nle]D[Nle]RCSASVECAQKCLAAIGSIFGKCMNKKCKC | 80 |
| | | YPR-NH₂ | |
| 80 | 82 | H-S[Nle]D[Nle]RCSASVECKQKCLKAIGSIFGKC[Nle]NK | 67 |
| | | KCKCYPR-OH | |
| 81 | 83 | H-P[Nle]D[Nle]RCSASVECKQKCLKAIGCIFGKC[Nle]NK | 81 |
| | | KCKCYPC-NH₂ | |
| 82 | 84 | H-S[Nle]D[Nle]RCSASVECKEKCLQAIGSIFGKC[Nle]NK | 82 |
| | | KCKCYPR-NH₂ | |
| 83 | 85 | H-P[Nle]D[Nle]RCSASVECKEKCL[homo-Gln]AIGSIFG | 83 |
| | | KC[Nle]NKKCKCYPR-NH₂ | |
| 84 | 86 | H-CSASVECKQKCLKAIGCIFGKC[Nle]NKKCKCYPC-NH₂ | 84 |
| 85 | 87 | H-S[Nle]D[Nle]RCSASVECKQKCLAAIGCIFGKC[Nle]NK | 85 |
| | | KCKCYPC-NH₂ | |
| 86 | 88 | H-P[Nle]D[Nle]RCSASVECKQKCLKAIGCIFGKC[Nle]NK | 81 |
| | | KCKCYPC-OH | |
| 87 | 91 | H-S[Nle]D[Nle]RCSASVECKQKCLKAIGSIFGKCMNKKCKC | 86 |
| | | [F(4-F)]PR-NH₂ | |
| 88 | 92 | H-S[Nle]D[Nle]RCSASVECKQKCLKAIGSIFGKCMNKKCKC | 87 |
| | | [F(4-NO₂)]PR-NH₂ | |
| 89 | 93 | H-S[Nle]D[Nle]RCSASVECKQKCLKAIGSIFGKCMNKKCKC | 88 |
| | | [F(4-CH₃)]PR-NH₂ | |
| 90 | 94 | H-[Nle]D[Nle]RCSASVECKQKCLKAIGSIFGKC[Nle]NKK | 89 |
| | | CKCYPR-OH | |
| 91 | 95 | H-NID[Nle]RCSASVECKQKCLKAIGSIFGKC[Nle]NKKCKC | 90 |
| | | YPR-OH | |
| 92 | 96 | H-PIE[Nle]RCSASVECKQKCLKAIGSIFGKC[Nle]NKKCKC | 91 |
| | | YPR-OH | |
| 93 | 99 | H-P[Nle]E[Nle]RCSASVECKQKCLLAIGSIFGKC[Nle]NK | 92 |
| | | KCKCYPR-OH | |
| 94 | 100 | H-PIDERCSASVECKQKCLAAIGSIFGKC[Nle]NKKCKCYPR- | 93 |
| | | OH | |
| 95 | 101 | H-PIE[Nle]RCSASVECKQKCLAAIGSIFGKC[Nle]NKKCKC | 94 |
| | | YPR-OH | |
| 96 | 102 | H-P[Nle]D[Nle]RCSASVECAQKCLAAIGSIFGKCMNKKCKC | 95 |
| | | YPR-OH | |
| 97 | 103 | H-P[Nle]E[Nle]RCSASVECAQKCLAAIGSIFGKC[Nle]NK | 96 |
| | | KCKCYPR-OH | |
| 98 | 104 | H-CSASVECKQKCLKAIGSIFGKC[Nle]NKKCKCYPR-NH₂ | 97 |
| 99 | 105 | Ac-CSASVECKQKCLKAIGSIFGKC[Nle]NKKCKCYPR-NH₂ | 97 |
| 100 | 106 | H-RCSASVECKQKCLKAIGSIFGKC[Nle]NKKCKCYPR-NH₂ | 98 |
| 101 | 107 | Ac-SKCSASVECKQKCLKAIGSIFGKC[Nle]NKKCKCYPR-NH₂ | 99 |
| 102 | 108 | Ac-LRCSASVECKQKCLKAIGSIFGKC[Nle]NKKCKCYPR-NH₂ | 100 |
| 103 | 114 | H-LRCSASVECKQKCLKAIGSIFGKC[Nle]NKKCKCYPR-OH | 100 |
| 104 | 115 | Ac-LRCSASVECKQKCLAAIGSIFGKC[Nle]NKKCKCYPR-OH | 101 |
| 105 | 116 | Ac-LRCSASVECKQKCLAAIGSIFGKCMNKKCKCYPR-OH | 102 |
| 106 | 117 | H-CSASVECKQKCLKAIGSIFGKCMNKKCKCYPR-OH | 103 |
| ShK | — | H-RSCIDTIPKSRCTAFQCKHSMKYRLSFCRKTCGTC-OH | 104 |
| ShK- | — | H-[phosphotyrosyl][8-Amino-3,6-dioxaoctanoyl] | 105 |
| 186 | | RSCIDTIPKSRCTAFQCKHSMKYRLSFCRKTCGTC-NH₂ | |
| Moka1 | — | H-INVKCSLPQQCIKPCKDAGMRFGKCMNKKCRCYS-OH | 106 |
| Vm24 | — | H-AAAISCVGSPECPPKCRAQGCKNGKCMNRKCKCYYC-NH₂ | 107 |

### Example 2: Kv1.3 Blocker Activity in FLIPR Thallium Assay

A human Kv1.3 voltage-gated K+ channel cell line was purchased from Perkin Elmer (#TDS-AX-010-C-1). The cell line is based on CHO-DUKX cells stably transfected with the human Kv1.3 voltage-gated K+ channel.

The cell line was grown in MEMα with nucleotides, GlutaMAX (Gibco #32571028), 10% Foetal Bovine Serum (FBS), 0.4 mg/ml Geneticin, 100 units/ml Penicillin, and 100 μg/ml Streptomycin and seeded at 10.000 cells/well in black poly-D-lysine-coated 96 well plates.

The FluxOR™ Potassium Ion Channel Assay (Invitrogen #F10016) was used to quantitate flux of thallium ions into the cells as a response to Kv1.3 activation with a stimulus buffer causes depolarization of the cell membrane, thereby generating a fluorescent signal, proportional to channel activity. The assay was performed as described by the assay kit manufacturer including a compound pre-incubation step (30 min, 37° C., 5% CO₂) in assay buffer containing 0.02% W/v casein from bovine milk (Sigma #C4765) prior to cell membrane depolarization using a final stimuli buffer composition corresponding to 0.2× FluxOR™ chloride-free buffer, 5 mM K₂SO₄, 1 mM Tl₂SO₄. The fluorescence responses were recorded and quantified (maximum response at 50 s, 37° C.) using the FLIPR® Tetra High Throughput Screening System (Molecular Devices, Inc.).

Data from test compounds eliciting an inhibition of thallium flux into the cell were normalized relative to the positive (ShK, 10 nM) and negative control (vehicle) to calculate the IC₅₀ from the concentration response curve using the 4-parameter logistic (4PL) nonlinear concentration response model based on the formula Y=Bottom+(Top−Bottom)/(1+10\hat{ }((LogIC50−X)*HillSlope)), where Y is percent inhibition, X is compound concentrations and Top, Bottom, Hill Slope, and IC50 are parameters fitted. Results are shown in Table 3 (n≥2). IC₅₀ can be regarded as a measure of potency of inhibition for the respective compound. The IC₅₀ value is a measure of the concentration of an inhibitor required to achieve half of that compound's maximal inhibition of ion channel activity in a given assay. A compound which has a lower IC₅₀-value at a particular ion channel than a reference compound can be considered to be a more active inhibitor, or a more potent inhibitor, than the reference compound.

TABLE 3

| Cpd No. | IC₅₀ (nM) |
|---|---|
| 1 | 0.12 |
| 2 | 0.12 |
| 3 | 0.13 |
| 4 | 0.15 |
| 5 | 0.18 |
| 6 | 33 |
| 7 | 0.28 |
| 8 | 0.26 |
| 9 | 4.6 |
| 10 | 0.33 |
| 11 | 0.22 |
| 12 | 0.45 |
| 13 | 0.11 |
| 14 | 0.15 |
| 15 | 0.24 |

### Example 3: Pharmacokinetic Characterisation

### Method

Sprague Dawley (males with a body weight of approximately 250-350 g) were given a single subcutaneous (s.c.) injection of each peptide to be tested.

Following s.c. administration of the selected compounds (dose 100 nmol/kg, dosing volume either 2 mL/kg), blood samples were drawn at 5 min, 15 min, 30 min, 60 min, 2 h, 3 h, 4 h, 6 h, 8 h post-dose. At each sampling time point, samples from the rats were drawn by sublingual bleeding. After last sampling the rats were sacrificed by O₂/CO₂ anaesthesia. The dosing vehicle was 10 mM phosphate, 0.8% NaCl, 0.05% Polysorbate 20 (pH 6.0).

Plasma samples were analyzed after solid phase extraction (SPE) by liquid chromatography mass spectrometry (LC-MS/MS). Mean plasma concentrations were used for calculation of the pharmacokinetic parameters using the non-compartmental approach in Phoenix WinNonlin 6.4 or a later version. Plasma terminal elimination half-life (T½) was determined as In(2)/λz where λz is the magnitude of the slope of the log linear regression of the log concentration versus time profile during the terminal phase. AUC_{inf} is the area under the plasma concentration—time curve extrapolated to infinity (AUC_{inf}=AUCₗₐₛₜ+Cₗₐₛₜ/λz, where Cₗₐₛₜ is the last observed plasma concentration). Cmax is the maximum observed concentration, occurring at Tmax. Results for selected compounds are shown in Table 4.

TABLE 4

| Cpd | Dose | AUC_{INF} | Cmax | T_{1/2} |
|---|---|---|---|---|
| No. | (nmol/kg) | (hr*nmol/L) | (nmol/L) | (hr) |
| 1 | 100 | 152 | 90.8 | 1.7 |
| 4 | 100 | 138 | 91.8 | 2.7 |
| 14 | 100 | 183 | 103 | 1.0 |

The pharmacokinetic characterization of compound 14 were performed according to the method described in example 6 of international patent application PCT/EP2020/076187. Results for compound 13 is shown in the below Table 5.

TABLE 5

| Cpd. | Dose | AUC_{INF} | Cmax | T_{1/2} |
|---|---|---|---|---|
| No. | (nmol/kg) | (hr*nmol/L) | (nmol/L) | (hr) |
| 13 | 70 | 26.8 | 16.5 | 1.7 |

### Example 4: Kv1.3 Selectivity in Patch Clamp Assay

Chinese Hamster Ovary (CHO) cell lines stably expressing exogenous human α-subunits of each potassium ion channel were grown and passaged under standard culture conditions.

The automated, chip-based planar patch clamp device QPatch® was used to quantitate the ionic currents. All recordings were made in the conventional whole-cell configuration after establishment of gigaohm seals. External recording solution contained (150 mM NaCl, 10 mM KCl, 10 mM HEPES, 1 mM MgCl₂, 3 mM CaCl₂), 10 mM Glucose, pH adjusted to 7.4 with NaOH) and Internal recording solution (20 mM KCl, 120 mM KF, 10 mM HEPES, 10 mM EGTA, 5 mM NaATP, pH adjusted to 7.2 with KOH). During experiments 0.1% (v/v) BSA was included as a vehicle in all external recording solutions. Currents were elicited from a holding potential of −80 mV using a voltage protocol, which shifted the voltage to 30 mV for 500 ms every 15 s.

Concentration-response relationships were established by cumulatively applying seven escalating concentrations of test sample to an individual cell with a recording period of 2 min per compound application.

The efficacy was determined as the mean charge for the last three sweeps at the end of each concentration application period from the cursor positions. The percent inhibition for each test dose application period was calculated as the reduction in mean cursor value (charge) relative to the cursor value measured at the end of the vehicle period and used to calculate the IC₅₀ from the concentration response curve.

Results are shown in Table 6.

TABLE 6

| | | | | | Selectivity ratio | | |
|---|---|---|---|---|---|---|---|
| Cpd | Potency IC₅₀ (nM) | | | | Kv1.1/ | Kv1.2/ | Kv1.6/ |
| No. | Kv1.1 | Kv1.2 | Kv1.3 | Kv1.6 | Kv1.3 | Kv1.3 | Kv1.3 |
| 1 | >3000 | 480 | 0.028 | >3000 | >100000 | 17000 | >100000 |
| 3 | >3000 | 380 | 0.064 | >3000 | >46000 | 6000 | >46000 |

Both compound 1 and 3 show very high selectivity for Kv1.3, as selectivity ratios of 6000 or higher towards Kv1.1, Kv1.2, and Kv1.6 were observed. Thus, at therapeutically relevant concentrations of compound 1 or 3, no detectable inhibition of these ion channels is expected.

Selectivity of reference compounds was also determined using the same method. Results are shown in Table 7.

TABLE 7

| Cpd | Potency IC₅₀ (nM) | | | | Selectivity | | |
|---|---|---|---|---|---|---|---|
| No. | Kv1.1 | Kv1.2 | Kv1.3 | Kv1.6 | Kv1.1/Kv1.3 | Kv1.2/Kv1.3 | Kv1.6/Kv1.3 |
| 15 | >3000 | 518 | 0.38 | >3000 | >7900 | 1364 | >7900 |
| 16 | >3000 | 97 | 1.50 | 1810 | >2000 | 65 | 1204 |
| 17 | >3000 | 409 | 0.27 | >3000 | >11300 | 1540 | >11300 |
| 18 | >3000 | 285 | 0.38 | >3000 | >7900 | 747 | >7900 |
| 19 | >3000 | 376 | 0.44 | >3000 | >6800 | 857 | >6800 |
| 20 | >3000 | 228 | 0.11 | >3000 | >27600 | 2093 | >27600 |
| 21 | >3000 | 133 | 0.24 | 2460 | >12600 | 561 | 10360 |
| 22 | >3000 | 66 | 0.42 | 912 | >7200 | 159 | 2182 |
| 23 | >3000 | 256 | 0.24 | >3000 | >12700 | 1080 | >12700 |
| 24 | >3000 | 45 | 0.22 | 435 | >13500 | 202 | 1955 |
| 25 | >3000 | 99 | 0.21 | 2527 | >14300 | 471 | 12045 |
| 26 | >3000 | 57 | 0.22 | 626 | >13700 | 259 | 2851 |
| 27 | >3000 | 181 | 0.29 | >3000 | >10200 | 615 | >10200 |
| 28 | 626 | 28 | 0.15 | 215 | 4280 | 191 | 1472 |
| 29 | >3000 | 67 | 0.09 | 363 | >34200 | 761 | 4144 |
| 30 | >3000 | 43 | 0.26 | >3000 | >11800 | 169 | >11800 |
| 31 | >3000 | 11 | 0.12 | 206 | >25900 | 94 | 1778 |
| 32 | >3000 | 16 | 0.20 | 252 | >15200 | 83 | 1272 |
| 33 | >3000 | 874 | 0.40 | >3000 | >7400 | 2163 | >7400 |
| 34 | >3000 | 666 | 0.14 | >3000 | >22000 | 4897 | >22000 |
| 35 | >3000 | 179 | 0.28 | >3000 | >10800 | 648 | >10800 |
| 36 | | | 0.38 | | | | |
| 38 | >3000 | 100 | 0.42 | 207 | >7100 | 237 | 490 |
| 39 | >3000 | 18 | 0.20 | 192 | >15000 | 88 | 963 |
| 40 | >3000 | 439 | 0.31 | >3000 | >9700 | 1422 | >9700 |
| 41 | >3000 | 338 | 0.19 | >3000 | >16100 | 1816 | >16100 |
| 43 | | | 0.15 | | | | |
| 44 | >3000 | 113 | 0.26 | 1829 | >11600 | 436 | 7073 |
| 45 | >3000 | 939 | 0.18 | >3000 | >17100 | 5362 | >17100 |
| 46 | >3000 | 109 | 0.16 | 316 | >18600 | 674 | 1954 |
| 49 | >3000 | 257 | 0.75 | >3000 | >4000 | 343 | >4000 |
| 50 | >3000 | 302 | 0.32 | 1395 | >9400 | 943 | 4359 |
| 51 | >3000 | 772 | 0.54 | 609 | >5600 | 1438 | 1134 |
| 52 | >3000 | >3000 | 0.37 | >3000 | >8200 | >8200 | >8200 |
| 53 | >3000 | 164 | 1.34 | 946 | >2200 | 123 | 707 |
| 54 | | | 0.27 | | | | |
| 55 | | | 0.25 | | | | |
| 56 | >3000 | 88 | 0.11 | >3000 | >27500 | 803 | >27500 |
| 57 | >3000 | 149 | 0.29 | >3000 | >10300 | 514 | >10300 |
| 58 | >3000 | 291 | 2.41 | >3000 | >1200 | 121 | >1200 |
| 59 | | | 0.12 | | | | |
| 60 | >3000 | 57 | 0.30 | 932 | >9900 | 187 | 3062 |
| 61 | >3000 | 145 | 0.34 | >3000 | >8800 | 426 | >8800 |
| 62 | >3000 | 449 | 0.26 | 2349 | >11600 | 1740 | 9102 |
| 63 | >3000 | 184 | 0.31 | 1231 | >9600 | 587 | 3929 |
| 64 | | | 0.25 | | | | |
| 65 | | | 0.14 | | | | |
| 66 | | | 0.18 | | | | |
| 67 | | | 0.32 | | | | |
| 70 | >3000 | 423 | 0.25 | >3000 | >12200 | 1720 | >12200 |
| 71 | >3000 | 373 | 0.11 | >3000 | >27700 | 3438 | >27700 |
| 75 | | | 0.13 | | | | |
| 76 | >3000 | 122 | 0.28 | >3000 | >10600 | 430 | >10600 |
| 78 | >3000 | 1149 | 0.21 | >3000 | >14600 | 5596 | >14600 |
| 79 | | | 0.16 | | | | |
| 81 | >3000 | 624 | 0.63 | 1222 | >4800 | 995 | 1949 |
| 90 | >3000 | 1859 | 0.17 | >3000 | >17800 | 11034 | >17800 |
| 91 | >3000 | 2063 | 0.27 | >3000 | >11300 | 7756 | >11300 |
| 92 | >3000 | 1758 | 0.39 | >3000 | >7800 | 4561 | >7800 |
| 14 | >3000 | 1117 | 0.33 | >3000 | >9100 | 3384 | >9100 |
| 13 | >3000 | 311 | 0.18 | >3000 | >16200 | 1689 | >16200 |
| 95 | >3000 | 1968 | 0.35 | >3000 | >8600 | 5630 | >8600 |
| 97 | >3000 | >3000 | 0.55 | >3000 | >5500 | 5500 | >5500 |
| 98 | >3000 | >3000 | 0.43 | >3000 | >7000 | >7000 | >7000 |
| 99 | >3000 | 384 | 0.24 | >3000 | >12400 | 1591 | >12400 |
| 100 | >3000 | 280 | 0.40 | 1302 | >7600 | 708 | 3290 |
| 101 | >3000 | 196 | 1.00 | 1451 | >3000 | 196 | 1451 |
| 102 | >3000 | 122 | 0.35 | 2456 | >8700 | 352 | 7096 |
| 106 | >3000 | 1832 | 2.09 | >3000 | >1400 | 878 | >1400 |
| ShK | 0.0021 | 11 | 0.017 | 0.19 | 0.12 | 644 | 11 |
| ShK- | 0.31 | 43 | 0.095 | 0.45 | 4.9 | 682 | 7.4 |
| 186 | | | | | | | |
| Moka1 | >3000 | 275 | 9.0 | >3000 | >300 | 31 | >300 |
| Vm24 | 0.097 | 4.7 | 0.15 | 21 | 0.65 | 31 | 139 |

### Example 5a: Inhibitory Activity of Kv1.3 Blocker Reference Compounds on Human PBMCs

Human peripheral blood mononuclear cells (PBMCs) were used to assess the effects of Kv1.3 blocker reference compounds on T-cell activation as determined by IL-2 (cytokine) release after stimulation with anti-CD3.

Human PBMCs were obtained from Precision for Medicine (Frederick, MD). Cells from 5 donors were used. Plate-bound anti-CD3 was used to stimulate bulk T cells in the PBMC preparations. Briefly, 96-well plates were coated with anti-CD3 antibody for 2 hrs at 37° C., using 50 μL of a 0.5 μg/mL anti-CD3 solution diluted in 1×PBS. Thereafter the plates were washed twice.

Kv1.3 blockers as shown in Table 8a were diluted in medium (RPMI 1640 with Glutamax-I containing 10% v/v Fetal Bovine Serum, 1% v/v penicillin-streptomycin solution) and added in a volume of 100 μL at concentrations ranging from 0.01 pM to 100 nM (tenfold dilutions). Cyclosporin A (1 ug/ml) and Vm24 peptide (100 nM) were used as positive controls. Finally, 1×105 PBMCs were added to each well in a volume of 100 μL, giving a final volume of 200 μL per well. The plates were incubated for 20-24 hours in a 37ºC/5% CO₂ incubator. After centrifugation of the plates, 25 μl supernatant was transferred to IL-2 detection plates (MSD Human IL-2 Tissue Culture Kit, cat #K151AHB-2) and IL-2 was measures as described by the manufacturer (Meso Scale Discovery, Rockville, Maryland, USA).

Results are shown in Table 8a as geometric mean of IC50 values obtained from anti-CD3 stimulated human PBMC assays. All values derive from at least 4 replicates.

TABLE 8a

| | anti-CD3 PBMC/IL-2 |
|---|---|
| Compound | IC50 [nM] |
| ShK-186 | 0.07 |
| 21 | 0.05 |
| 22 | 0.4 |
| 25 | 0.1 |
| 39 | 0.1 |

Incubation with anti-CD3 antibody activated hPBMC and addition of Kv1.3 blockers resulted in dose-dependent reduction in the IL-2 secretion. On average the IC₅₀ values (as calculated from IL-2 release) of the test compounds were in the range of 0.05 nM to 0.4 nM. This was comparable to the IC₅₀ observed with ShK186 (IC₅₀ is 0.07 nM) and about 10-100 fold lower than the IC₅₀ of Moka1 which was less potent in inhibiting IL-2 secretion.

There is no significant difference between the test compounds and ShK186. ShK186 and test compounds were all significantly lower than Moka1.

Cyclosporine blocked CD3 induced IL-2 release completely in all experiments.

### Example 5b: Inhibitory Activity of Kv1.3 Blocker Reference Compounds on Human PBMCs

Human peripheral blood mononuclear cells (PBMCs) were used to assess the effects of Kv1.3 blocker reference compounds on T-cell activation as determined by IL-2 release after stimulation with anti-CD3.

Human PBMCs were obtained from Precision for Medicine (Frederick, MD). Cells from 5 donors were used. Plate-bound anti-CD3 was used to stimulate bulk T cells in the PBMC preparations. Briefly, 96-well plates were coated with anti-CD3 antibody for app. 16 hours at 5° C., using 50 μl of a 1 μg/ml anti-CD3 solution diluted in PBS. Thereafter the plates were washed twice.

Kv1.3 blockers were subsequently diluted in medium (RPMI 1640 with Glutamax-I containing, 10% v/v Fetal Bovine Serum, 1% v/v penicillin-streptomycin Solution) and added in a volume of 50 μl. The compounds indicated in Table 8b were used at concentrations ranging from 0.3 pM to 1000 nM (half log dilutions, starting concentrations varying). Cyclosporin A (1 μg/ml) and Vm24 peptide (100 nM) were used as positive controls.

Finally, 50.000 PBMCs in the same medium were added to each well in a volume of 50 μl, giving a final volume of 100 μl per well. The plates were incubated for 20-24 hours in a 37° C./5% CO₂ incubator. After centrifugation of the plates, 25 μl supernatant was transferred to IL-2 detection plates (MSD Human IL-2 Tissue Culture Kit, cat #K151AHB-2) and IL-2 was measures as described by the manufacturer (Meso Scale Discovery, Rockville, Maryland, USA).

Results are shown in Table 8b as geometric mean of IC50 values obtained from anti-CD3 stimulated human PBMC assays. All values derive from at least 6 replicates.

TABLE 8b

| | anti-CD3 PBMC/IL-2 |
|---|---|
| Compound | IC50 [nM] |
| ShK-186 | 0.04 |
| 17 | 0.06 |
| 15 | 0.09 |
| 45 | 0.03 |
| 28 | 0.06 |
| 52 | 0.07 |

Incubation with anti-CD3 antibody activated hPBMC and addition of the Compounds of this invention resulted in dose-dependent reduction in the IL-2 secretion.

The average IC₅₀ values (as calculated from IL-2 release) of the test compounds were in the range of 0.01 nM to 0.09 nM as shown in Table 8b. This was comparable to the IC₅₀ observed with ShK186 (IC₅₀ is 0.05 nM). This assay was performed using different donors than those used for Example 5a, so identical values for the Shk-186 in the two sets of experiments are not expected.

Cyclosporine blocked anti-CD3 induced IL-2 release completely in all experiments.

### Example 6: Inhibitory Activity of Kv1.3 Blocker Reference Compounds in Rat Whole Blood

Rat whole blood was used to assess the potency of Kv1.3 blocker reference compounds on T-cell activation as determined by IL-17A release after stimulation with thapsigargin. Addition of thapsigargin results in activation of a signalling cascade ending up in activation of T cell proliferation and cytokine production where the Kv1.3 ion channel plays a key role, so activity of Kv1.3 blockers in primary cells can be measured in this experimental system.

Rat whole blood was obtained from healthy, naïve Lewis or Sprague-Dawley rats that were terminally bleed from the heart using Sodium Heparin blood sampling tubes for collection. Test compounds were diluted to 4× final testing concentrations in assay buffer (DMEM+GlutaMAX), GlutaMAX is a medium comprising 3.97 mM L-alanine-L-glutamine (Gibco Cat #61965026) supplemented with 25 mM HEPES buffer, 1 mM Sodium Pyruvate, 100 units/ml Penicillin, 100 μg/ml Streptomycin and 0.05% Casein from bovine milk (Sigma-Aldrich)) and 25 μl was added to wells of a 96 well plate. Then 50 μl whole rat blood was added and incubated for minimum 5 minutes at room temperature to allow compound binding. Then 25 μl 40 μM thapsigargin diluted in assay buffer was added to all wells of the assay plates to activate the cells, followed by incubation for 24 Hr at 37ºC/5% CO₂ in a humidified box. The assay plates were centrifuged for 10 min at 300 g at 4ºC and the supernatants were transferred to new plates. The concentrations of IL-17A released to the supernatants were measured using a Rat IL-17A ELISA Kit (Abcam Cat #ab214028) as recommended by the manufacturer. Samples were diluted 2.5-fold by transferring 20 μl of the supernatants to wells on ELISA plates containing 30 μl buffer 75BS from the detection kit.

Data from test compounds eliciting an inhibition of IL-17A were normalised relative to full thapsigargin activation (no blocker added) and no activation controls (addition of assay buffer instead of thapsigargin) to calculate the IC₅₀ from the concentration response curve.

Results are shown in Table 9, expressed as IC₅₀, with standard deviation (IC₅₀_SD). All values are derived from at least 2 replicates. The biological effects ex vivo show a correlation with the potency of the compounds.

TABLE 9

| CPD NO | IC₅₀ | IC_{50 —}SD |
|---|---|---|
| 15 | 0.17 | 0.036 |
| 17 | 1.0 | 0.31 |
| 22 | 0.88 | 0.32 |
| 23 | 3.2 | 0.017 |
| 24 | 0.56 | 0.2 |
| 25 | 1.2 | 0.72 |
| 26 | 0.82 | 0.81 |
| 27 | 2.8 | 2.5 |
| 28 | 0.57 | 0.24 |
| 30 | 0.61 | 0.33 |
| 31 | 0.68 | 0.47 |
| 32 | 0.53 | 0.21 |
| 33 | 0.76 | 0.23 |
| 34 | 0.41 | 0.021 |
| 35 | 0.86 | 0.31 |
| 36 | 0.75 | 0.24 |
| 37 | 0.70 | 0.40 |
| 38 | 2.1 | 0.89 |
| 39 | 2.5 | 2.1 |
| 40 | 1.2 | 0.7 |
| 41 | 0.77 | 0.56 |
| 42 | 2.8 | 1.8 |
| 45 | 0.98 | 0.60 |
| 46 | 0.91 | 0.26 |
| 47 | 0.99 | 0.24 |
| 48 | 1.1 | 0.47 |
| 49 | 1.4 | 0.64 |
| 50 | 0.79 | 0.28 |
| 51 | 0.83 | 0.4 |
| 52 | 0.63 | 0.039 |
| 53 | 1.0 | 0.65 |
| 56 | 0.69 | 0.21 |
| 62 | 0.44 | 0.080 |
| 63 | 0.68 | 0.19 |
| 64 | 1.2 | 0.71 |
| 65 | 1.1 | 0.011 |
| 66 | 0.47 | 0.35 |
| 67 | 0.98 | 0.076 |
| 68 | 0.93 | 0.72 |
| 69 | 2.7 | 0.54 |
| 70 | 0.79 | 0.15 |
| 71 | 0.30 | 0.11 |
| 72 | 0.47 | 0.086 |
| 73 | 0.52 | 0.019 |
| 74 | 1.6 | 1.1 |
| 75 | 0.86 | 0.13 |
| 76 | 0.55 | 0.25 |
| 77 | 0.39 | 0.28 |
| 80 | 0.52 | 0.059 |
| 81 | 0.81 | 0.13 |
| 82 | 0.69 | 0.38 |
| 83 | 1.4 | 1.1 |
| 84 | 1.1 | 1.3 |
| 85 | 0.73 | 0.28 |
| 86 | 1.1 | 1.2 |
| 87 | 0.34 | 0.07 |
| 88 | 1.3 | 1.0 |
| 89 | 0.54 | 0.31 |
| 90 | 0.55 | 0.42 |
| 91 | 1.2 | 0.57 |
| 92 | 1.8 | 1.2 |
| 14 | 0.43 | 0.42 |
| 13 | 0.34 | 0.21 |
| 93 | 0.5 | 0.37 |
| 94 | 0.87 | 0.84 |
| 95 | 0.61 | 0.34 |
| 96 | 0.32 | 0.22 |
| 97 | 1.2 | 1.5 |
| 98 | 1.6 | 0.94 |
| 99 | 0.88 | 0.011 |
| 100 | 0.52 | 0.34 |
| 101 | 0.36 | 0.13 |
| 102 | 0.37 | 0.049 |
| 103 | 2.1 | 2.5 |
| 104 | 0.9 | 0.3 |
| 105 | 0.36 | 0.24 |
| 106 | 1.9 | 1 |

### Example 7: Effect of Kv1.3 Blocker Reference Compound Treatment in the Keyhole Limpet Hemocyanin (KLH) Ear Inflammation Model in Rats

A classical delayed-type hypersensitivity (DTH) reaction was elicited in one ear of rats. Briefly, male Lewis rats aged 8-10 weeks were immunized on day −7 with 200 μL keyhole limpet hemocyanin (KLH) (from Sigma, cat. no. H7017) (4 mg/mL) emulsified in complete Freund's adjuvant (CFA) (Difco, cat. no. 263810) subcutaneously (SC) at the base of the tail. On day 0 the rats were challenged intradermally with 40 μL KLH/NaCl 0.9% (2 mg/mL) in the left ear. After the ear challenge the rats develop a T-cell dependent inflammation in the left KLH challenged ear. The right ear remains uninflamed and serves as control.

The ability of Kv1.3 blocker reference compound treatment to reduce the DTH ear swelling response was investigated by comparing the response in rats (n=8-10/gr) treated with vehicle to that of rats treated with a Kv1.3 blocker. Vehicle or Kv1.3 blocker dissolved in vehicle was administered SC (2 mL/kg) 24 hrs prior to KLH ear challenge. The test dose of Kv1.3 blocker was 50, 70 or 100 nmol/kg. Test vehicle was 10 mM phosphate, 0.8% w/v NaCl, 0.05% w/v polysorbate20, pH 6. Cyclosporine (CsA) was included as positive study control in all experiments. Cyclosporine (Sandimmune Neooral® 100 mg/ml oral solution, Novartis) was administered per os (10 mg/kg) one hour prior to KLH ear challenge and again 6 hours after KLH ear challenge.

As primary read-out of efficacy, the Area Under Curve (AUC) of Δ ear thickness (mm) was calculated for each animal from 0-48 hours post induction of the ear DTH reaction, where the change (D) was calculated as: Left ear thickness-right ear thickness. These results were then used to calculate % inhibition of ear thickness by Kv1.3 blocker treatment: % inhibition: ((1−(individual Δ AUC Kv1.3 blocker/average ΔAUC vehicle group))×100. Results were calculated as % inhibition+/−standard deviation (SD), and are shown in Table 10 and Table 11.

TABLE 10

| | Dose | | | | |
|---|---|---|---|---|---|
| Exp | (nmol/kg) | Cpd. 17 | Cpd. 45 | Cpd. 56 | CsA* |
| #1 | 70 | 38.8 (+/−9.7) | 46.9 (+/−10.9) | | 71.4 (+/−4.8) |
| #2 | 70 | 25.0 (+/−12.5) | 27.7 (+/−11.5) | | 76.8 (+/−13.4) |
| #3 | 50 | 37.3 (+/−13.8) | 22.2 (+/−11.1) | | 65.1 (+/−4.5) |
| #4 | 100 | | 41.9 (+/−12.5) | 25.1 (+/−6.3) | 63.4 (+/−5.9) |

TABLE 11

| | Dose | | | | | |
|---|---|---|---|---|---|---|
| Exp. | (nmol/kg) | Cpd. 91 | Cpd. 14 | Cpd. 95 | Cpd. 97 | CsA |
| #5 | 100 | 39.6 | | | | 67.1 |
| | | (+/−8.3) | | | | (+/−7.9) |
| #6 | 100 | | 42.5 | 44.5 | | 55.3 |
| | | | (+/−14.0) | (+/−5.2) | | (+/−5.5) |
| #7 | 100 | | | | 48.9 | 67.9 |
| | | | | | (+/−8.6) | (+/−5.4) |
| #8 | 100 | | 37.7 | | | 57.8 |
| | | | (+/−10.0) | | | (+/−2.8) |

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in biochemistry, molecular biology or related fields are intended to be within the scope of the following claims.

1. An ion channel blocker which has Kv1.3 inhibitor activity, or a pharmaceutically acceptable salt thereof, comprising a variant of the sequence

| (SEQ ID NO: 1) | |
|---|---|
| | QMDMRCSASVECKQKCLKAIGSIFGKCMNKKCKCYPR, |

wherein the variant differs from SEQ ID NO: 1 by up to ten substitutions, insertions or deletions in total,
wherein at least one amino acid in position 7, 8, 9, 10 or 11 of SEQ ID NO: 1 is substituted with an amino acid having a positively charged side chain and/or an amino acid having an aromatic side chain, and wherein the variant does not comprise any of the following sequences:
SEQ ID NO: 2
NMDMRCKASVECKQKCLKAIGSIFGKCMNKKCKCYPR
SEQ ID NO: 3
NMDMRCSASRECKQKCLKAIGSIFGKCMNKKCKCYPR
SEQ ID NO: 4
N[Nle]D[Nle]RCRASVECKQKCLKAIGSIFGKC[Nle]NKKCKCYPR
SEQ ID NO: 5
N[Nle]D[Nle]RCSHSVECKQKCLKAIGSIFGKC[Nle]NKKCKCYPR
SEQ ID NO: 6
N[Nle]D[Nle]RCSASKECKQKCLKAIGSIFGKC[Nle]NKKCKCYPR
SEQ ID NO: 7
P[Nle]E[Nle]RCFASVECKQKCLAAIGSIFGKC[Nle]NKKCKCYPR
SEQ ID NO: 8
P[Nle]E[Nle]RCSYSVECKQKCLAAIGSIFGKC[Nle]NKKCKCYPR
SEQ ID NO: 9
P[Nle]E[Nle]RCSAFVECKQKCLAAIGSIFGKC[Nle]NKKCKCYPR

2. The ion channel blocker of any of the preceding claims wherein any substitutions or deletions in the variant of SEQ ID NO: 1 are at amino acid positions selected from positions 1-5, 7-11, 13-15, 17-23, 25, 28-31, 33 and 35-37 of SEQ ID NO: 1, preferably at amino acid positions selected from positions 1-5, 7-11, 13, 15, 18, 28 and 30 of SEQ ID NO: 1.

3. The ion channel blocker of any of the preceding claims, wherein the ion channel blocker has an IC₅₀ for human Kv1.3 potassium channel of 50 nM or less, such as 20 nM or less, such as 10 nM or less, such as 5 nM or less, such as 2 nM or less, such as 1 nM or less, such as 0.5 nM or less, such as 0.4 nM or less, such as 0.3 nM or less, such as 0.2 nM or less.

4. The ion channel blocker of any of the preceding claims wherein
the amino acid having a positively charged side chain is selected from the group consisting of H, K, hK, R, Orn, 2,3-diaminopropanoyl, 2,4-diaminobutanoyl, 2-amino-3-guanidinopropionyl, and 4-amino-phenylalanine (F(4-NH₂)), preferably from H, K, R, Orn, 2,3-diaminopropanoyl, 2-amino-3-guanidinopropionyl, and 2,4-diaminobutanoyl; and/or
the amino acid having an aromatic side chain is selected from the group consisting of F, W and Y, preferably Y.

5. The ion channel blocker of any of the preceding claims wherein
the amino acid in position 7 of SEQ ID NO: 1 is substituted with an amino acid having a positively charged side chain or an aromatic side chain, preferably H, K, R, Orn, 2,3-diaminopropanoyl, 2-amino-3-guanidinopropionyl, 2,4-diaminobutanoyl or Y;
the amino acid in position 8 of SEQ ID NO: 1 is substituted with an amino acid having a positively charged side chain or an aromatic side chain, or the amino acid in position 8 of the variant is the same amino acid as position 8 in SEQ ID NO: 1, namely A;
the amino acid in position 9 of SEQ ID NO: 1 is substituted with an amino acid having a positively charged side chain or an aromatic side chain, preferably K, Orn or 2,3-diaminopropanoyl;
the amino acid in position 10 of SEQ ID NO: 1 is substituted with an amino acid having a positively charged side chain or an aromatic side chain, preferably R; and/or
the amino acid in position 11 of SEQ ID NO: 1 is substituted with an amino acid having a positively charged side chain or an aromatic side chain, preferably R.

6. The ion channel blocker of any of the preceding claims wherein, in the variant of SEQ ID NO: 1,
the amino acid at position 1 is N, P or Q, or is deleted,
the amino acid at position 2 is M or Nle, or is deleted,
the amino acid at position 3 is D or E, or is deleted,
the amino acid at position 4 is M or Nle, or is deleted,
the amino acid at position 5 is R or is deleted,
the amino acid at position 13 is A or K,
the amino acid at position 15 is K or S,
the amino acid at position 18 is A or K,
the amino acid at position 28 is M or Nle, and/or
the amino acid at position 30 is G or K.

7. The ion channel blocker of any of the preceding claims wherein, in the variant of SEQ ID NO: 1,
the amino acids at positions 1-5 consist of the amino acid sequence P[Nle]E[Nle]R,
the amino acid at position 13 is A or K,
the amino acid at position 15 is K or S,
the amino acid at position 18 is A,
the amino acid at position 28 is Nle, and
the amino acid at position 30 is G or K.

8. The ion channel blocker of any of the preceding claims wherein the variant of SEQ ID NO: 1 comprises or consists of one of the following sequences:

| | SEQ |
|---|---|
| | ID |
| Sequence | NO |
| P[Nle]E[Nle]RC[2,4-Diaminobutanoyl]ASVECKQKC | 10 |
| LAAIGSIFGKC[Nle]NKKCKCYPR | |
| P[Nle]E[Nle]RC[Orn]ASVECKQKCLAAIGSIFGKC[Nle] | 11 |
| NKKCKCYPR | |
| P[Nle]E[Nle]RC[2-Amino-3-guanidinopropionyl] | 12 |
| ASVECKQKCLAAIGSIFGKC[Nle]NKKCKCYPR | |
| P[Nle]E[Nle]RCHASVECKQKCLAAIGSIFGKC[Nle]NKKC | 13 |
| KCYPR | |
| P[Nle]E[Nle]RCYASVECKQKCLAAIGSIFGKC[Nle]NKKC | 14 |
| KCYPR | |
| P[Nle]E[Nle]RCSA[Orn]VECKQKCLAAIGSIFGKC[Nle] | 15 |
| NKKCKCYPR | |
| P[Nle]E[Nle]RC[2,3-Diaminopropanoyl]ASVECKQK | 16 |
| CLAAIGSIFGKC[Nle]NKKCKCYPR | |
| P[Nle]E[Nle]RCSA[2,3-Diaminopropanoyl]VECKQK | 17 |
| CLAAIGSIFGKC[Nle]NKKCKCYPR | |
| P[Nle]E[Nle]RC[2,4-Diaminobutanoyl]ASVECAQSC | 18 |
| LAAIGSIFGKC[Nle]NGKCKCYPR | |
| P[Nle]E[Nle]RCRASVECAQSCLAAIGSIFGKC[Nle]NGKC | 19 |
| KCYPR | |
| P[Nle]E[Nle]RCSASRECAQSCLAAIGSIFGKC[Nle]NGKC | 20 |
| KCYPR | |

9. The ion channel blocker of any of the preceding claims wherein the variant comprises or consists of one of the following compounds:

| Cpd | | SEQ |
|---|---|---|
| No. | Sequence | ID NO |
| 1 | H-P[Nle]E[Nle]RC(1)[2,4-Diaminobutanoyl]ASVEC(2)KQKC | 10 |
| | (3)LAAIGSIFGKC(1)[Nle]NKKC(2)KC(3)YPR-OH | |
| 2 | H-P[Nle]E[Nle]RC(1)[Orn]ASVEC(2)KQKC(3)LAAIGSIFGKC(1) | 11 |
| | [Nle]NKKC(2)KC(3)YPR-OH | |
| 3 | H-P[Nle]E[Nle]RC(1)[2-Amino-3-guanidinopropionyl]ASV | 12 |
| | EC(2)KQKC(3)LAAIGSIFGKC(1)[Nle]NKKC(2)KC(3)YPR-OH | |
| 4 | H-P[Nle]E[Nle]RC(1)HASVEC(2)KQKC(3)LAAIGSIFGKC(1) | 13 |
| | [Nle]NKKC(2)KC(3)YPR-OH | |
| 5 | H-P[Nle]E[Nle]RC(1)YASVEC(2)KQKC(3)LAAIGSIFGKC(1)[Nle] | 14 |
| | NKKC(2)KC(3)YPR-OH | |
| 6 | H-P[Nle]E[Nle]RC(1)SA[Orn]VEC(2)KQKC(3)LAAIGSIFGKC(1) | 15 |
| | [Nle]NKKC(2)KC(3)YPR-OH | |
| 7 | H-P[Nle]E[Nle]RC(1)[2,3-Diaminopropanoyl]ASVEC(2)KQKC | 16 |
| | (3)LAAIGSIFGKC(1)[Nle]NKKC(2)KC(3)YPR-OH | |
| 8 | H-P[Nle]E[Nle]RC(1)SA[2,3-Diaminopropanoyl]VEC(2)KQKC | 17 |
| | (3)LAAIGSIFGKC(1)[Nle]NKKC(2)KC(3)YPR-OH | |
| 9 | H-P[Nle]E[Nle]RC(1)[2,4-Diaminobutanoyl]ASVEC(2)AQSC | 18 |
| | (3)LAAIGSIFGKC(1)[Nle]NGKC(2)KC(3)YPR-OH | |
| 10 | H-P[Nle]E[Nle]RC(1)[2,4-Diaminobutanoyl]ASVEC(2)AQSC | 18 |
| | (3)LAAIGSIFGKC(1)[Nle]NGKC(2)KC(3)YPR-[NH2] | |
| 11 | H-P[Nle]E[Nle]RC(1)RASVEC(2)AQSC(3)LAAIGSIFGKC(1)[Nle] | 19 |
| | NGKC(2)KC(3)YPR-[NH2] | |
| 12 | H-P[Nle]E[Nle]RC(1)SASREC(2)AQSC(3)LAAIGSIFGKC(1)[Nle] | 20 |
| | NGKC(2)KC(3)YPR-[NH2] | |

10. A nucleic acid encoding an ion channel blocker as defined in any of the preceding claims.

11. An expression vector comprising a nucleic acid according to claim 10.

12. A host cell comprising a nucleic acid according to claim 10 or an expression vector according to claim 11 and capable of expressing an ion channel blocker as defined in any of claims 1 to 9.

13. A method of synthesising an ion channel blocker according to any of claims 1 to 9, the method comprising:
(a) synthesising the ion channel blocker by means of solid-phase or liquid-phase peptide synthesis methodology and recovering the peptide thus obtained;
(b) expressing the ion channel blocker from a nucleic acid construct that encodes the ion channel blocker and recovering the expression product; or
(c) expressing a precursor peptide from a nucleic acid construct that encodes the precursor peptide sequence, recovering the expression product, and modifying the precursor peptide to yield the ion channel blocker.

14. A pharmaceutical composition comprising an ion channel blocker or pharmaceutically acceptable salt according to any one of claims 1 to 9.

15. An ion channel blocker, a pharmaceutically acceptable salt or a pharmaceutical composition according to any of the preceding claims for use in a method of medical treatment, preferably treatment of an inflammatory condition or disorder.
